(19) United States
(12) Patent Application Publication
(10) Pub. No.: US 2005/0074814 A1
(43) Pub. Date: Apr. 7, 2005

(54) Relaxin3-GPCR135 complexes and their production and use

(76) Inventors: Jingcai Chen, Beijing (CN); Chester Kuei, Murrieta, CA (US); Changlu Liu, San Diego, CA (US); Timothy W. Lovenberg, San Diego, CA (US); Rannar Sillard, Stockholm (SE); Steven W. Sutton, Carlsbad, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/786,478

(22) Filed: Feb. 25, 2004

Related U.S. Application Data
(60) Provisional application No. 60/451,702, filed on Mar. 4, 2003.

(51) Int. Cl. G01N 33/53; G06F 19/00; G01N 33/48; G01N 33/50

(52) U.S. Cl. 435/7.1; 530/350; 702/19

(57) ABSTRACT

A GPCR135/relaxin3 complex is described, which is useful as an assay reagent for screening for modulators of GPCR135 receptor activity.

### FIELD OF THE INVENTION

The present invention relates to the ligand-and-receptor complexes of GPCR135 and relaxin3. The invention also relates to processes of preparing such complexes and methods of their use. The invention further relates to an isoform of the rat GPCR135 receptor.

### BACKGROUND OF THE INVENTION

GPCR135

G-protein coupled receptors (GPCRs) are transmembrane receptor proteins that are responsible for the transduction of a diverse array of extracellular signals, including hormones, neurotransmitters, peptides, lipids, ions, light, odorants, nucleotides, fatty acid derivatives, and other chemical mediators. See, e.g., WIPO Publication No. WO02/00719. GPCRs are of particular importance to drug discovery because they have been established as excellent drug targets: they are the targets of 50% of marketed drugs. An increasing number of diseases have been found to be associated with GPCRs. Drugs targeting GPCRs have been used to treat a wide range of disorders from cardiovascular to gastro-intestinal to CNS and others (Wilson et al., 1998, British J. of Pharmacology 125:1387-1392).

The GPCR-mediated signal transduction event is often initiated upon binding of a specific ligand to the GPCR. Each GPCR is composed of an extracellular N-terminal domain, seven distinct transmembrane segments, and an intracellular C-terminal domain. Binding of the ligand to the extracellular N-terminal domain of GPCR results in a conformational change that leads to activation of intracellular heterotrimeric GTP-binding proteins (G proteins) associated with the GPCR. These activated G proteins in turn mediate a variety of intracellular responses that regulate the cell physiology. Therefore, the ligand provides means of elucidating the physiological function of the GPCR as well as methods of screening for compounds that regulate the signal transduction activity of the GPCR.

At present, only about 200 GPCRs are classified as known GPCRs that are activated by around 70 known ligands. Through sequence analyses, it was discovered that GPCRs belong to one of the largest superfamilies of the human genome: evaluated at over 1000 genes encoding GPCRs (Civelli et al., 2001, Trends in Neurosciences 24:230-237). A large number of putative GPCRs are described as orphan receptors because their natural ligands are unknown. Some of these uncharacterized orphan GPCRs may be useful as therapeutic targets. The identification of the specific ligand to a GPCR is the key to harnessing the potential therapeutic benefits of these orphan GPCRs (Howard et al., 2001, Trends in Pharmacological Sciences 22:132-140).

GPCR135 is an orphan receptor that has been found to be expressed almost exclusively in the central nerve system. GPCR135 has also been referred to as Somatostatin- and Angiotensin-Like Peptide Receptor (SALPR)(Matsumoto et al., 2000, Gene 248:183-189). In humans, GPCR135 shares about 35% and 31% amino acid sequence similarity with the receptor of somatostatin (SSTR5) and angiotensin (AT1), respectively. However, transient expression of GPCR135 in Cos-1 cells did not produce any binding sites for somatostatin or angiotensin II, indicating that these ligands do not bind to GPCR135. The putative amino acid sequence of GPCR135 suggests that its ligand is peptidergic in nature. The GPCR135 mRNA is expressed in discrete brain region such as substantia nigra and pituitary at low levels, implying that a previously unidentified neuropeptide is possibly the GPCR135 ligand (Matsumoto et al., 2000, supra).

Relaxin3

Human relaxin3 (H3) and its mouse equivalent (M3) have been identified from the Celera genomic database as relaxin-related peptides based on sequence homology (Bathgate et al., 2002, J. Biol. Chem. 277: 1148-1157). Either gene encodes a putative prohormone sequence incorporating the classic two-chain, three cysteine-bounded structure of the relaxin/insulin family and the RXXXRXX(I/V)motif in the B-chain that is essential for relaxin receptor binding. Compared with other members of the insulin/relaxin family, H3 and M3 are grouped under a separate branch on the phylogenic tree, indicating that the evolution of these particular relaxins diverged from the other relaxins, such as relaxin1 or relaxin2, which primarily function as hormones of pregnancy. Therefore, relaxin3 may have a function distinct from that of relaxin1 or 2. Recent studies demonstrated that relaxin3 activates LGR7, a leucine-rich repeat-containing orphan GPCR that may have roles in reproductive, brain, renal, cardiovascular, and other functions (Hsu et al., (2002) Science 295, 671-674; Sudo et al., 2003, J. Biol. Chem, 278: 7855-7862). Although little is known about the distribution of relaxin3 mRNA in human tissues due to its low level of expression, M3 mRNA was detected in the mouse brain, particularly in the pons/medulla in cells encompassing the pars ventromedialis of the dorsal tegmental nucleus and the dorsal part of the reticular nucleus of female C57BLK6J mice. This suggests possible role of relaxin3 as a neuropeptide in mammalian brains (Bathgate et al., 2002, supra).

### SUMMARY OF THE INVENTION

It has now been discovered that relaxin3 is the natural ligand for GPCR135.

In one general aspect, the invention therefore relates to a receptor-ligand complex comprising a receptor component containing GPCR135 or an active fragment of GPCR135 bound to a ligand component containing relaxin3 or an active fragment of relaxin3, wherein at least one of the receptor and ligand components is in a substantially pure form.

In other general aspects, the invention relates to: an isolated polynucleotide having a nucleotide sequence encoding an amino acid sequence of SEQ ID NO: 15, or a complement thereof; an isolated polypeptide having such amino acid sequence; a vector consisting of a polynucleotide encoding such polypeptide; and a recombinant host cell comprising such vector.

An additional general aspect of the invention relates to a process of producing a relaxin3 from a recombinant cell, comprising: constructing a DNA molecule encoding a relaxin3 propeptide modified by insertion of a peptide linkage forming a protease cleavage site at at least one location selected from the peptide junction between chains A and C and the peptide junction between chains C and B of the relaxin3 propeptide; constructing a first vector expressing the modified relaxin3 propeptide; constructing a second vector expressing a protease for cleaving the modified relaxin3 propeptide at the inserted protease cleavage site; introducing both the first vector and the second vector into a host cell; and growing the host cell so that both the modified relaxin3 propeptide and the protease are expressed, whereby the protease efficiently cleaves the modified relaxin3 propeptide at the inserted peptide linkage.

Further general aspects of the invention relate to assay or screening methods. For example, one general method of identifying a compound that increases or decreases a biological activity of a GPCR135/relaxin3 complex comprises: contacting a test sample comprising a compound, and a buffering solution with an assay reagent comprising a receptor-ligand complex of the invention; determining the biological activity of the receptor-ligand complex; and comparing the measurement obtained in that determination with a control measurement wherein the receptor-ligand complex has been contacted with the buffering solution. Another general assay method of identifying a compound that binds to GPCR135 or an active fragment thereof comprises: contacting GPCR135 or an active fragment thereof with a test compound and with a labeled relaxin3 or an active fragment thereof; and determining the amount of the labeled relaxin3 or active fragment thereof that binds to the GPCR135 or active fragment thereof, and then comparing that amount with a control measurement wherein the GPCR135 or active fragment thereof has been contacted with the labeled relaxin3 or active fragment thereof in the absence of test compound. A further general assay relates to a method for identifying a compound that binds GPCR135 and mimics relaxin3, comprising: contacting a test compound with an assay reagent comprising GPCR135 or an active fragment thereof; determining a biological activity of the GPCR135 or active fragment thereof; and comparing this result with that of a control measurement wherein the GPCR135 or an active fragment thereof was contacted with relaxin3 or an active fragment thereof in the absence of the test compound.

Other aspects, features and advantages of the invention will be apparent from the following disclosure, including the detailed description of the invention and its preferred embodiments and the appended claims.

### BRIEF DESCRIPTION OF THE DRAWINGS

FIG. 1 illustrates amino acid sequence comparison among human, mouse, and rat GPCR135. The rat sequence shown is rat GPCR135s.

FIG. 2 illustrates GPCR135 ligand activity in different rat tissues. Different rat tissues were extracted using ethanol/HCl method. The resulting tissue extracts at various dilutions were used as ligand in GTPγS binding assays with isolated human GPCR135 expressing cell membranes as the source of the receptor.

FIG. 3 shows the molecular weight characterization of GPCR135 ligand. Crude rat brain ethanol/HCl extract was run through a HPLC-gel filtration column. Fractions were collected at one fraction per minute. Each fraction was assayed for GPCR135 ligand activity in GTPγS binding assay. In a parallel experiment, peptides and nucleotides with known molecular weights were run at the sample condition to serve as the standards.

FIG. 4 illustrates amino acid sequence comparison among human, mouse, rat relaxin3 (R3) and the GPCR135 ligand purified from porcine brain.

FIG. 5 illustrates GPCR135 ligand activity in the cell culture medium from COS cells transfected with human relaxin3. Cell culture medium from COS cells transfected with human relaxin3 (COS7 HR3) or without human relaxin3 (COS7 Ct) was concentrated by ion exchange and C-18 column. The concentrated cell culture media were diluted at various dilutions, and tested for GPCR135 ligand activity using GTPγS binding assays.

FIG. 6 demonstrates that relaxin3 stimulates Ca²⁺ mobilization in HEK293 cells co-expressing GPCR135 and Gqi5. HEK293 cells, either untransfected (293 Control) or transfected with Gqi5 (293/Gqi5) alone, human GPCR135 (GPCR135) alone, or co-transfected by human GPCR135 and Gqi5 (GPCR135/Gqi5), were used for Ca²⁺ mobilization assays under stimulation of human relaxin3. Ligand stimulated intracellular Ca²⁺ mobilization was monitored using FLIPR.

FIG. 7 shows that ¹²⁵I-relaxin3 specifically binds GPCR135. COS7 cells, either untransfected (NC) or transfected with pcDNA3.1 (the vector control), human GPCR135 (hGPCR135), mouse GPCR135 (mGPCR135), rat GPCR135 long form (rGPCR135L), and rat GPCR135 short form (rGPCR135s) respectively, were used for binding assays using ¹²⁵I-labeled human relaxin3 either in the present (“Competition”) or absence (“Total Binding”) of 1 □M of unlabeled human relaxin3 as the competitor.

FIG. 8 shows that binding of relaxin3 to GPCR135 decreases intracellular cAMP concentration from a cAMP accumulation assays. CHO-K1 cells stably expressing human GPCR135 (GPCR135) and un-transfected CHO-K1 cells (Control) were used for cAMP accumulation assays. Cells were treated with different stimulants as indicated. cAMP accumulated in the cells was extracted by HCl and the concentration of cAMP was measured by a ELISA kit (NEN) using ¹²⁵I-labeled cAMP and Flash plates.

### DETAILED DESCRIPTION OF INVENTION AND ITS PREFERRED EMBODIMENTS

All publications cited below are hereby incorporated by reference. Unless defined otherwise, all technical and scientific terms used herein have the same meaning as commonly understood to one of ordinary skill in the art to which this invention pertains.

The following are abbreviations that are at times used in this specification below:
bp=base pair
Ca²⁺=calcium ion
cAMP=cyclic adenosine monophosphate
cDNA=complementary DNA
CNS=central nervous system
kb=kilobase; 1000 base pairs
kDa=kilodalton; 1000 dalton
GPCR=G protein coupled receptor
G protein=GTP-binding protein
GTP=guanosine 5′-triphosphate
nt=nucleotide
PAGE=polyacrylamide gel electrophoresis
PCR=polymerase chain reaction
SDS=sodium dodecyl sulfate
SiRNA=small interfering RNA
UTR=untranslated region.

The terms “including,” “comprising” and “containing” are used herein in their open, non-limiting sense.

“An activity”, “a biological activity”, or “a functional activity” of a polypeptide or nucleic acid refers to an activity exerted by a polypeptide or nucleic acid molecule as determined in vivo, or in vitro, according to standard techniques. Such activities can be a direct activity, such as an association with or an enzymatic activity on a second protein, or an indirect activity, such as a cellular signaling activity mediated by interaction of the protein with a second protein.

An exemplary biological activity of relaxin3 is its ability to bind GPCR135 and initiate the signal transduction event conducted by GPCR135. An exemplary biological activity of GPCR135 is that, upon binding to relaxin3, GPCR135 activates a chain of events that alters the concentration of intracellular signaling molecules (second messenger molecule), such as cyclic AMP and calcium via activating G-protein, which has a high affinity to GTP. These intracellular signaling molecules in turn alter the physiology and behavior of the cell.

A “clone” is a population of cells derived from a single cell or common ancestor by mitosis. A “cell line” is a clone of a primary cell that is capable of stable growth in vitro for many generations.

A “disorder related to the relaxin3 and GPCR135 complex” means a disorder or disease associated with overactivity or insufficient activity of the relaxin3 and GPCR135 complex, and conditions that accompany such a disorder or disease. The term “overactivity” refers to either increased expression of the ligand and receptor complex, or increased biological activities of the complex. The term “insufficient activity” refers to either decreased expression of the ligand and receptor complex, or decreased biological activities of the complex.

“Genetic variant” or “variant” means a specific genetic variant which is present at a particular genetic locus in at least one individual in a population and that differs from the wild type.

As used herein, a “GPCR135” or “receptor component” refers to a polypeptide that: (1) has greater than about 60% amino acid sequence identity, to human GPCR135, also called SALPR (Matsumoto et al., 2000. Gene 248:183-189, GenBank Protein Accession No.: BAA93001); (2) is capable of binding to antibodies, e.g., polyclonal or monoclonal antibodies, raised against a human GPCR135 protein described herein; or (3) is encoded by a polynucleotide that specifically hybridizes under stringent hybridization conditions to a nucleic acid molecule having a sequence that has greater than about 60% nucleotide sequence identity to human GPCR135 cDNA (GenBank nucleotide Accession No.: D88437). “Stringent hybridization conditions” has the meaning known in the art, as described in Sambrook et al., Molecular Cloning: A Laboratory Manual, Second Edition, Cold Spring Harbor Laboratory, Cold Spring Harbor, N.Y., (1989).

In preferred embodiments, the receptor component is a polypeptide having greater than 65, 70, 75, 80, 85, 90, or 95 percent amino acid sequence identity to human GPCR135. In other preferred embodiments, the receptor component is a polypeptide encoded by a polynucleotide that specifically hybridizes under stringent hybridization conditions to a nucleic acid molecule having a sequence that has greater than 65, 70, 75, 80, 85, 90, or 95 percent nucleotide sequence identity to human GPCR135 cDNA.

Exemplary GPCR135 receptor components include orthologs that have been identified in human (SEQ ID NO: 12, GenBank Accession Numbers: BAA93001, Q9NSD7, and NP_{—}057652), rat (SEQ ID NO: 14, and SEQ ID NO: 15, 85.9% sequence identity to that of human), mouse (SEQ ID NO: 13, 86.4% sequence identity to that of human), and other animals, including pig and monkey. An “active fragment of GPCR135” means any fragment of a GPCR135 protein that maintains the biological activity of a GPCR135, such as binding to a mammalian relaxin3 and activating a G-protein. The protein or cDNA sequence of human GPCR135 has been disclosed in patent applications, WO00/23111, WO00/24891, WO01/48189, WO01/62797, WO01/74904, and WO01/85791.

As used herein, the term “host cell” refers to a cell that contains a DNA molecule either on a vector or integrated into a cell chromosome. A host cell can be either a native host cell that contains the DNA molecule endogenously, or a recombinant host cell as defined infra.

An “isolated” nucleic acid molecule is one that is substantially separated from nucleic acid molecules with differing nucleic acid sequences. Embodiments of the isolated nucleic acid molecule of the invention include cDNA, genomic DNA and RNA, preferably of human origin.

An “isolated” or “purified” protein or biologically active fragment or portion thereof is substantially free of cellular material or other contaminating proteins from the cell or tissue source from which the protein is produced and isolated, or substantially free of chemical precursors or other chemicals when the protein is chemically synthesized. For example, protein that is substantially free of cellular material can include preparations of protein having less than about 30%, 20%, 10%, or 5% (by dry weight) of contaminating proteins. When the protein or biologically active portion thereof is recombinantly produced, it is also preferably substantially free of culture medium, i.e., culture medium representing less than about 20%, 10%, or 5% of the volume of the protein preparation. When the protein is produced by chemical synthesis, it is preferably substantially free of chemical precursors or other chemicals, i.e., it is separated from chemical precursors or other chemicals that are involved in the synthesis of the protein. Accordingly such preparations of the protein have less than about 30%, 20%, 10%, or 5% (by dry weight) of chemical precursors or compounds other than the polypeptide of interest.

Isolated biologically active polypeptides can have several different physical forms. The isolated polypeptide can exist as a full-length nascent or unprocessed polypeptide, or as partially processed polypeptides or combinations of processed polypeptides. The full-length nascent polypeptide can be post-translationally modified by specific proteolytic cleavage events that result in the formation of fragments of the full-length nascent polypeptide. A fragment, or physical association of fragments, can have the biological activity associated with the full-length polypeptide; however, the degree of biological activity associated with individual fragments can vary.

The term “linker region” or “linker domain” or similar such descriptive terms as used herein refers to one or more polynucleotide or polypeptide sequences that are used in the construction of a cloning vector or fusion protein. The function of a linker region can include introduction of cloning sites into the nucleotide sequence, introduction of a flexible component or space-creating region between two protein domains, or creation of an affinity tag to facilitate a specific molecule interaction. A linker region can be introduced into a fusion protein, if desired, during polypeptide or nucleotide sequence construction.

The term “nucleic acid” as used herein refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single- or double-stranded. However, linkages may include any of the linkages known in the art, including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like. Furthermore, the term “nucleic acid sequences” contemplates the complementary sequence and includes any nucleic acid sequence that is substantially homologous to the both the nucleic acid sequence and its complement.

“Polynucleotide” refers to a linear polymer of at least 2 nucleotides joined together by phosphodiester bonds and may comprise ribonucleotides or deoxyribonucleotides.

“Polymorphism” refers to a set of genetic variants at a particular genetic locus among individuals in a population.

“Polypeptide” refers to any peptide or protein comprising two or more amino acids joined to each other in a linear chain by peptide bonds. As used herein, the term refers both to short chains, which also commonly are referred to in the art as, e.g., peptides, oligopeptides and oligomers, and to longer chains, which generally are referred to in the art as proteins, of which there are many types. It will be appreciated that polypeptides often contain amino acids other than the 2.0 amino acids commonly referred to as the 20 naturally occurring amino acids, and that many amino acids, including the terminal amino acids, can be modified in a given polypeptide, either by natural processes, such as processing and other post-translational modifications, but also by chemical modification techniques known to the art. Even the common modifications that occur naturally in polypeptides are too numerous to list exhaustively here, but they are well described in basic texts and in more detailed monographs, as well as in research literature, and are therefore within the purview of persons of ordinary skill in the art. Among the known modifications which can be present in polypeptides of the present invention are, to name an illustrative few, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Several common modifications, such as glycosylation, lipid attachment, sulfation, gamma-carboxylation of glutamic acid residues, hydroxylation and ADP-ribosylation, are described in many basic texts, including PROTEINS—STRUCTURE AND MOLECULAR PROPERTIES, 2nd Ed., T. E. Creighton, W. H. Freeman and Company, New York (1993). Many detailed reviews are also available on this subject, such as those provided by Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al. (1990), Meth. Enzymol. 182, 626-646; and Rattan et al., “Protein Synthesis: Posttranslational Modifications and Aging”, (1992) Ann. N.Y. Acad. Sci. 663, 48-62.

It will be appreciated, as is known and as noted above, that polypeptides are not always entirely linear. For instance, polypeptides can be post-translationally modified, including via natural processing or through human manipulation. Circular, branched and branched-circular polypeptides can be synthesized by non-translation natural processes and by entirely synthetic methods as well. Modifications can occur anywhere in a polypeptide, including the peptide backbone, the amino acid side-chains and the amino or carboxyl termini. For example, blockage of the amino or carboxyl group or both in a polypeptide by a covalent modification is common in naturally occurring and synthetic polypeptides, and such modifications can be present in polypeptides of the present invention. For instance, the amino terminal residue of polypeptides made in E. coli or other cells, prior to proteolytic processing, almost invariably will be N-formylmethionine. During post-translational modification of the peptide, a methionine residue at the NH₂-terminus can be deleted. Accordingly, this invention contemplates the use of both the methionine-containing and the methionineless amino terminal variants of the protein.

The modifications that occur in a polypeptide often will be a function of how it is made. For polypeptides made by expressing a cloned gene in a host, for instance, the nature and extent of the modifications in large part will be determined by the host cell posttranslational modification capacity and the modification signals present in the polypeptide amino acid sequence. For instance, as is known, glycosylation often does not occur in bacterial hosts such as, for example, E. coli. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to express efficiently mammalian proteins having native patterns of glycosylation, inter alia. Similar considerations apply to other modifications. It will be appreciated that the same type of modification can be present in the same or varying degree at several sites in a given polypeptide. Also, a given polypeptide can contain many types of modifications. In general, as used herein, the term “polypeptide” encompasses all such modifications, including those that are present in polypeptides synthesized recombinantly by expressing a polynucleotide in a host cell.

“Prohormone convertase (PC)” refers to a family of Ca²⁺-dependent serine proteases, all of which possess homology to the bacterial endoproteases subtilisin (bacteria) and yeast kexin. This family, also known as furin/paired basic amino-acid-cleaving enzyme (PACE), includes, e.g., PC1/PC3, PC2, PC4, PACE4, PC5/PC6, and PC7/PC8/lymphoma proprotein convertase, and SKI-1. They share a degree of amino-acid identity of 50-75% within their catalytic domains (for a review on PCs, see Seidah et al., 1999, Brain Res, 27;848(1-2):45-62).

“Promoter” means a regulatory sequence of DNA that is involved in the binding of RNA polymerase to initiate transcription of a gene. Promoters are often upstream (“5″ to”) the transcription initiation site of the gene. A “gene” is a segment of DNA involved in producing a peptide, polypeptide, or protein, including the coding region, non-coding regions preceding (“5″ UTR”) and following (“3″ UTR”) coding region, as well as intervening non-coding sequences (“introns”) between individual coding segments (“exons”). “Coding” refers to the representation of amino acids, start and stop signals in a three base “triplet” code.

A “recombinant host cell” is a cell that has been transformed or transfected by an exogenous DNA sequence. As used herein, a cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed or transfected cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cells containing the exogenous DNA. Recombinant host cells may be prokaryotic or eukaryotic, including bacteria such as E. coli, fungal cells such as yeast, mammalian cells such as cell lines of human, bovine, porcine, monkey and rodent origin, and insect cells such as Drosophila and silkworm derived cell lines. It is further understood that the term “recombinant host cell” refers not only to the particular subject cell, but also to the progeny or potential progeny of such a cell. Because certain modifications can occur in succeeding generations due to either mutation or environmental influences, such progeny may not, in fact, be identical to the parent cell, but are still included within the scope of the term as used herein.

A “recombinant polypeptide” refers to a polypeptide produced by recombinant DNA techniques; i.e., produced from cells transformed by an exogenous DNA construct encoding the desired polypeptide. A “synthetic polypeptide” refers to that prepared by chemical synthesis.

A “relaxin3” or “ligand component” refers to a polypeptide that: (1) has greater than about 60% amino acid sequence identity to human relaxin3 (Bathgate et al., 2002. J. Biol. Chem. 277: 1148-1157, GenBank Protein Accession No.: NP_{—}543140); (2) is capable of binding to antibodies, e.g., polyclonal or monoclonal antibodies, raised against a human Relaxin3 protein described herein; or (3) is encoded by a polynucleotide that specifically hybridizes under stringent hybridization conditions to a nucleic acid molecule having a sequence that has greater than about 60% nucleotide sequence identity to human relaxin3 cDNA (GenBank nucleotide Accession No: NM_{—}080864).

In preferred embodiments, the ligand component is a polypeptide having greater than 65, 70, 75, 80, 85, 90, or 95 percent amino acid sequence identity to human relaxin3. In other preferred embodiments, the ligand component is a polypeptide encoded by a polynucleotide that specifically hybridizes under stringent hybridization conditions to a nucleic acid molecule having a sequence that has greater than 65, 70, 75, 80, 85, 90, or 95 percent nucleotide sequence identity to human relaxin3 cDNA.

Exemplary relaxin3 ligands include relaxin3 orthologs that have been identified in human (GenBank protein Accession No.: NP_{—}543140), rat (Burazin et al., 2002, J. Neurochem. 82: 1553-1557, GenBank protein Accession No. NP_{—}733767, 76.4% sequence identity to that of human,), mouse (Bathgate et al., 2002. J. Biol. Chem. 277: 1148-1157, GenBank protein accession number XP_{—}146603, 78.7% sequence identity to that of human), and other animals, including pig and monkey. A “relaxin3” includes all three forms of relaxin3: 1) the relaxin3 pre-propeptide or precursor, an intracellular polypeptide consisting of the signal sequence and the relaxin3 propeptide sequence; 2) the relaxin3 propeptide, a secreted polypeptide having sequences for chains A, C, and B of relaxin3 linked by protease cleavage sites; and 3) mature relaxin3 peptide, a secreted protein consisting of relaxin3 polypeptide chains A and B, linked by disulfide bridges. An “active fragment of relaxin3” includes any fragments of a relaxin3 protein that maintains a biological activity of relaxin3, such as binding to a GPCR135. The protein or cDNA sequence of human relaxin3 has been disclosed in WIPO Publication Nos. WO01/68862, WO01/81562, and WO02/22802. The protein or cDNA sequence of rat or mouse relaxin3 has been disclosed in WIPO Publication No. WO01/81562.

A “second messenger response of a cell” refers to cellular response of the cell mediated through activation of a GPCR135 upon binding of a relaxin3. It includes, e.g., signal transduction event as well as change in intracellular concentration of a second messenger molecule, such as proton (pH), calcium, or cAMP.

“Sequence” means the linear order in which monomers occur in a polymer, for example, the order of amino acids in a polypeptide or the order of nucleotides in a polynucleotide.

“Sequence identity or similarity”, as known in the art, is the relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences.

To determine the percent identity or similarity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same or similar amino acid residue or nucleotide as the corresponding position in the second sequence, then the molecules are identical or similar at that position. The percent identity or similarity between the two sequences is a function of the number of identical or similar positions shared by the sequences (i.e., % identity=# of identical positions/total # of positions (e.g., overlapping positions)×100). In one embodiment, the two sequences are the same length.

Both identity and similarity can be readily calculated. In calculating percent identity, only exact matches are counted. Methods commonly employed to determine identity or similarity between sequences include, e.g., those disclosed in Carillo et al. (1988), SIAM J. Applied Math. 48, 1073. Preferred methods to determine identity are designed to give the largest match between the sequences tested. Methods to determine identity and similarity are codified in computer programs.

A preferred example of a mathematical algorithm utilized for the comparison of two sequences is the algorithm of Karlin et al. (1990), Proc. Natl. Acad. Sci. USA 87:2264-2268, modified as in Karlin et al. (1993), Proc. Natl. Acad. Sci. USA 90:5873-5877. Such an algorithm is incorporated into the NBLAST and XBLAST programs of Altschul et al. (1990), J. Mol. Biol 215:403-410. To obtain gapped alignments for comparison purposes, Gapped BLAST can be utilized as described in Altschul et al. (1997), Nucleic Acids Res. 25:3389-3402. Alternatively, PSI-Blast can be used to perform an iterated search, which detects distant relationships between molecules. When utilizing BLAST, Gapped BLAST, and PSI-Blast programs, the default parameters of the respective programs (e.g., XBLAST and NBLAST) can be used. See, e.g., http://www.ncbi.nlm.nih.gov. Additionally, there is the FASTA method (Atschul et al. (1990), J. Molec. Biol. 215, 403), which can also be used.

Another preferred example of a mathematical algorithm useful for the comparison of sequences is the algorithm of Myers et al. (1988), CABIOS 4:11-17. Such an algorithm is incorporated into the ALIGN program (version 2.0), which is part of the GCG sequence alignment software package (Devereux et al. (1984), Nucleic Acids Research 12(1), 387).

The term “substantially similar” as used herein in reference to a polynucleotide or polypeptide sequence includes the identical sequence as well as deletions, substitutions or additions thereto that result in a modified sequence that maintains any biologically active portion and possesses any of the conserved motifs thereof.

The term “subject” as used herein refers to an animal who is the object of treatment, observation or experiment. Preferably, the subject is a mammal, more preferably a human.

The term “a substantially pure form” as used herein in reference to a receptor or ligand component means a polypeptide useful as a biological reagent that is essentially free of contaminating matter that would interfere with ligand-receptor binding. Substantially pure forms of polypeptides may be produced by, e.g., isolation, purification, peptide synthesis, or recombinant expression. In an exemplary embodiment, the ligand component is relaxin3 produced by isolation or recombinant expression and the receptor component is GPCR135 expressed on the surface of a whole cell. In another embodiment, both the receptor and ligand components are in substantially pure form so that the resulting complex can be used in applications such as x-ray crystallography to yield a co-crystal structure that may be employed in conformational studies or computational modeling to aid in the design of drugs useful in treating disorders mediated by modulation (e.g., agonism or antagonism) of relaxin3/GPCR135 interaction.

The term “tag” as used herein refers to an amino acid sequence or a nucleotide sequence encoding an amino acid sequence that facilitates isolation, purification or detection of a protein containing a tag. A variety of such tags are known to those skilled in the art and are suitable for use in the present invention. Suitable tags include, e.g., HA peptide, polyhistidine peptides, biotin/avidin, and a variety of antibody epitope binding sites.

The term “therapeutically effective amount” as used herein means that amount of active compound or pharmaceutical agent that elicits the biological or medicinal response in a tissue system, animal or human that is being sought by a researcher, veterinarian, medical doctor or other clinician, such as alleviation of the symptoms of the disease or disorder being treated. Methods are known in the art for routinely determining therapeutically effective doses for pharmaceutical compositions.

The term “vector” refers to a nucleic acid molecule capable of transporting another nucleic acid to which it has been linked. One type of vector is a “plasmid”, which refers to a circular double-stranded DNA loop into which additional DNA segments can be inserted. Another type of vector is a viral vector wherein additional DNA segments can be inserted. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors—expression vectors—are capable of directing the expression of genes to which they are operably linked. In general, vectors of utility in recombinant DNA techniques are often in the form of plasmids. However, the invention is intended to include other forms of vectors, such as viral vectors (e.g., replication defective retroviruses, adenoviruses and adeno-associated viruses), which serve equivalent functions.

Ligand/Receptor Complex

In preferred embodiments of the ligand-and-receptor complex of the invention, the ligand component is a relaxin3 that is originated from a human, a mouse, a rat, or another animal. Preferably, the ligand component is a relaxin3 that has been recombinantly expressed.

In another preferred embodiment of the ligand-and-receptor complex of the invention, the ligand component is labeled with a detectable agent, such as a radio-isotope or a fluorescent molecule. The labeling method varies with the type of labeling agent employed, and is known to those skilled in the art. For instance, labeling can be accomplished by replacing one of the atoms of the ligand molecules with a corresponding radioactive isotope. A hydrogen atom could be replaced with: tritium, ³H; a carbon atom can be replaced with carbon-14, ¹⁴C; or a strontium atom can be replaced with strontium-38, ³⁸Sr. In another exemplary labeling process, rather than replacing the atoms of the ligand with a radioactive isotope, an isotope can be added to the ligand molecule. Such radioactive isotopes include, for example, iodine-125, ¹²⁵I; and iron-59, ⁵⁹Fe. In yet another exemplary labeling process, labeling can be carried out by using an appropriate radio-labeled precursor, such as methionine-35 (³⁵S) or phosphate-33 (³³P, for protein phosphorylation), during the synthesis of the peptide either in vivo or in vitro. Preferably, the ligand component of this invention is labeled with iodine-125, ¹²⁵I.

Preferably, the ligand and receptor complex of the invention comprises a GPCR135 that is originated from a human, a mouse, or a rat, or another animal. More preferably, the receptor component is a GPCR135 having an amino acid sequence selected from SEQ ID NO: 12, SEQ ID NO: 13, SEQ ID NO: 14, and SEQ ID NO: 15, and fragments thereof.

In one preferred embodiment, the GPCR135 is expressed on the cell surface of a GPCR135 host cell, preferably a recombinant GPCR135 host cell. In another preferred embodiment, the GPCR135 is associated with isolated cell membranes from a GPCR135 host cell, preferably from a recombinant GPCR135 host cell. In yet another preferred embodiment, the receptor component of the complex is a fragment of the GPCR135 capable of binding to a relaxin3.

The ligand component of the complex can be either a full-length relaxin3 or an active fragment thereof that is still capable of binding to a GPCR135.

Any suitable methods to constitute a ligand and receptor complex known to the skilled artisan may be used to form the complex. In general, the method comprises mixing a sample comprising the ligand with a sample comprising the receptor.

The sample comprising the ligand can be tissue or cell extract containing the ligand, or purified ligand. This sample can be prepared from a natural source of the ligand, i.e., an endogenous host cell or tissue for the ligand of warm-blooded animals inclusive of human. Preferably, the sample comprising the ligand is prepared from a recombinant host cell that expresses increased amount of the ligand. A recombinant host cell for the ligand may be constructed by introducing into the cell a DNA molecule capable of expressing the functional ligand. Exemplary methods on construction of expression vectors as well as recombinant host cells for relaxin3 are described herein (see Example 4). A method for recombinant production of relxain3 is described infra.

In the production from the tissues or cells of human or other warm-blooded animals, the ligand polypeptide can be purified and isolated by a process comprising homogenizing the tissue or cells of human or other warm-blooded animal, extracting the homogenate with an acid or another suitable extracting agent, and isolating the polypeptide from the extract, e.g., via a combination of chromatographic procedures, such as reversed-phase chromatography, ion-exchange chromatography, affinity chromatography, etc. Exemplary methods are provided herein on preparing rat brain extract (see Example 2), and purifying relaxin3 from the porcine brains (see Example 3).

The ligand polypeptide in the present invention can also be produced by known procedures for peptide synthesis. The methods for peptide synthesis may be any of solid-phase synthesis and liquid-phase synthesis techniques. Thus, the desired peptide can be produced by condensing a partial peptide or amino acid capable of constituting the protein with the residual part thereof and, when the product has a protective group, removing the protective group. Methods for condensation and deprotection during peptide synthesis are described in literature, for example, in: (1) M. Bodanszky and M. A. Ondetti, Peptide Synthesis, Interscience Publishers, New York, 1966; (2) Schroeder and Luebke, The Peptide, Academic Press, New York, 1965; (3) Nobuo Izumiya et al., Fundamentals and Experiments in Peptide Synthesis, Maruzen, 1975; (4) Haruaki Yajima and Shumpei Sakakibara, Biochemical Experiment Series 1, Protein Chemistry IV, 205, 1977; and (5) Haruaki Yajima (ed.), Development of Drugs-Continued, 14, Peptide Synthesis, Hirokawa Shoten.

After the peptide synthesis reaction, the protein product can be purified and isolated by a suitable combination of conventional purification techniques, such as solvent extraction, column chromatography, liquid chromatography, and recrystallization. Where the protein isolated is in a free form, it can be converted to a suitable salt by a known method. Conversely, where the isolated product is a salt, it can be converted to the free peptide by a known method.

The amide of polypeptide can be obtained by using a resin for peptide synthesis that is suited for amidation. Exemplary resins include chloromethyl resin, hydroxymethyl resin, benzhydrylamine resin, aminomethyl resin, 4-benzyloxybenzyl alcohol resin, 4-methylbenz-hydrylamine resin, PAM resin, 4-hydroxymethylmethylphenyl-acetamidomethyl resin, polyacrylamide resin, 4-(2′,4′-dimethoxyphenyl-hydroxymethyl) phenoxy resin, and 4-(2′,4′-dimethoxyphenyl-Fmoc aminoethyl)phenoxy resin. Using such a resin, amino acids whose α-amino groups and functional groups of side-chain have been suitably protected are condensed on the resin according to the sequence of the objective peptide by various condensation techniques that are known to those skilled in the art. At the end of the series of reactions, the peptide or the protected peptide is separated from the resin and the protective groups are removed to obtain the objective polypeptide.

The sample comprising the GPCR135 receptor can comprise intact host cells with the receptor expressed on the cell-surface, isolated cell membranes from host cells of the receptor, or purified fragment of the receptor that is capable of binding to the ligand. Although an endogenous host cell for GPCR135 receptor can be used, a recombinant host cell expressing an increased amount of GPCR135 on the cell surface is preferred. An exemplary method of constructing a recombinant host cell for human GPCR135 is described in Example 1.

It is known that a GPCR binds to its ligand with its extracellular domain. Such a binding domain can be identified by various methods known to those skilled in the art, such as sequence analyses, protein-protein interaction analyses, protein structural analyses, or a combination of these methods. For example, the ligand binding domain in metabotropic glutamate receptors has been identified as a Venus flytrap module (VFTM) in its extracellular domain (O'Hara et al., 1993, Neuron, 11(1): 41-52; David et al., 1999, J Biol Chem, 274: 33488-33495). In a preferred embodiment, the relaxin3 binding domain of the GPCR135 can be first identified using the above methods, and such a binding domain can be recombinantly expressed, purified and used in forming a complex of the invention.

Rat GPCR135

One general aspect of the invention pertains to an isoform of a GPCR135, the rat GPCR135s. Two isoforms of rat GPCR135 have been cloned and identified in the invention: a longer isoform rat GPCR1351 and a shorter isoform rat GPCR135s. The rat GPCR1351 has the amino acid sequence of SEQ ID NO: 14, and is identical to SEQ ID NO: 1 of WIPO Publication No. WO00/24891. The rat GPCR135s has the amino acid sequence of SEQ ID NO: 15, and lacks the first seven amino acid residues at the N-terminal end of the longer form. Recombinantly expressed rat GPCR135s and GPCR1351 have similar activities in radio-ligand binding or GTPγS binding assays (Example 1), indicating that both rat isoforms have GPCR135 activities.

In one embodiment according to this general aspect, the present invention provides an isolated nucleic acid molecule encoding a rat GPCR135s consisting of the amino acid sequence of SEQ ID NO: 15, or a complement thereof. In another embodiment, the nucleic acid molecule encodes the amino acid sequence of SEQ ID NO: 15.

A nucleic acid molecule which is a “complement” of a given nucleotide sequence is one which is sufficiently complementary to the given nucleotide sequence that it can hybridize to the given nucleotide sequence, thereby forming a stable duplex under high stringency or stringent hybridization conditions. Exemplary high stringency or stringent hybridization conditions include: 50% formamide, 5×SSC and 1% SDS incubated at 42° C., or 5×SSC and 1% SDS incubated at 65° C., with a wash in 0.2×SSC and 0.1% SDS at 65° C.

In a preferred embodiment, the invention provides an isolated nucleic acid molecule consisting essentially of the nucleotide sequence of SEQ ID NO: 11, or a complement thereof. In a more preferred embodiment, the nucleic acid molecule has the nucleotide sequence of SEQ ID NO: 11.

The invention provides an isolated nucleic acid molecule consisting of a nucleic acid molecule which is a degenerate variant of rat cDNA as set forth in SEQ ID NO: 11, or a complement thereof. It is known that more than one genetic codon can be used to encode a particular amino acid, and therefore the amino acid sequence of rat GPCR135s as depicted in SEQ ID NO: 15 can be encoded by any of a set of similar DNA molecules. Only one member of the set will be identical to the cDNA sequence as set forth in SEQ ID NO: 11; however, all variants hereinafter referred to as “degenerate variants” are contemplated to be within the scope of this invention. Herein, a nucleic acid molecule bearing one or more alternative codons which encode a polypeptide with amino acid sequence set forth as SEQ ID NO: 15 is referred to as a degenerate variant of rat cDNA as set forth in SEQ ID NO: 11.

Particularly preferred in this regard are natural allelic variants of rat GPCR135s nucleic acid molecules. DNA sequence polymorphisms that lead to changes in the amino acid sequence can exist within a population of animals, i.e., a population of rats. Such genetic polymorphisms can exist among individuals within a population due to natural allelic variation. An “allele” is one of a group of genes which occur alternatively at a given genetic locus. Such natural allelic variations can typically result in 1-5% variance in the nucleotide sequence of a given gene. Alternative alleles can be identified by sequencing the gene of interest in a number of different individuals, or by using hybridization probes to identify the same genetic locus in a variety of individuals.

Further particularly preferred in this regard are nucleic acid molecules having any and all such nucleotide variations that are not known to occur naturally which encode polypeptides having properties that are different than, but still maintain the functional activity of, the naturally occurring rat GPCR135s protein. DNA sequences can be altered manually so as to code for a peptide having properties that are different from those of the naturally occurring peptide. Methods of altering the DNA sequences include site-directed mutagenesis, chimeric substitution, and gene fusions. Site-directed mutagenesis is used to change one or more DNA residues that can result in a silent mutation, a conservative mutation, or a nonconservative mutation. Chimeric genes are prepared by swapping domains of similar or different genes to replace domains in the rat GPCR315s gene. Similarly, fusion genes can be prepared that add domains to the rat GPCR135, such as an affinity tag to facilitate identification and isolation of the gene and protein.

The variants of the rat GPCR135s nucleic acid molecule of the invention are capable of hybridizing to SEQ ID NO: 11 under high stringent hybridization condition.

Cells or tissues that possess the rat GPCR135s transcript, preferably high levels of the transcript, are suitable for the isolation of rat GPCR135s cDNA or mRNA. Selection of suitable cDNA source can be done by screening for the presence of the rat GPCR135s transcript in cell extracts or in whole cells by nucleotide hybridization or RT-PCR analysis using primers that hybridize specifically to the rat GPCR135s transcript as depicted in SEQ ID NO: 11. A preferred source for isolation of the shorter isoform of rat GPCR135s nucleic acid is the rat brain.

Any of a variety of procedures known in the art can be used to isolate the nucleic acid molecule of the invention. For example, using cDNA or genomic DNA libraries, or total mRNA from the suitable cells identified above as a template and appropriate oligonucleotide as primers, a nucleic acid molecule of the invention can be amplified according to standard PCR amplification techniques. The nucleic acid so amplified from PCR can be cloned into an appropriate vector and characterized by DNA sequence analysis. The ordinarily skilled artisan will appreciate that oligonucleotides comprising at least 12 contiguous nucleotides of SEQ ID NO: 11 are particularly useful as primers. The primers can be prepared by standard synthetic techniques, e.g., using an automated DNA synthesizer.

Another method to isolate a nucleic acid molecule of the invention is to probe a genomic or cDNA library, or total mRNA with one or more natural or artificially designed probes using procedures recognized by those skilled in the art. See, e.g., “Current Protocols in Molecular Biology”, Ausubel et al. (eds.), Greene Publishing Association and John Wiley Interscience, New York, 1989, 1992. The ordinarily skilled artisan will appreciate that oligonucleotides comprising at least 12 contiguous nucleotides of SEQ ID NO: 11 are particularly useful probes. Preferred probes will have at least 30 bases. Particularly preferred probes will have 50 or less bases. Such probes can be and are preferably labeled with an analytically detectable reagent to facilitate identification of the probe. Useful reagents include, e.g., radioisotopes, fluorescent dyes, or enzymes capable of catalyzing the formation of a detectable product. The probes enable the ordinarily skilled artisan to isolate complementary copies of genomic DNA, cDNA or RNA polynucleotides encoding rat GPCR135s. Similar routine procedures can also be used to isolated nucleic acid molecules encoding GPCR315s proteins from other animal sources or to screen such sources for related sequences, e.g., additional members of the family, type and/or subtype, including transcriptional regulatory and control elements as well as other stability, processing, translation and tissue specificity-determining regions from 5′ and/or 3′ regions relative to the coding sequences disclosed herein.

Another method to prepare nucleic acid molecules corresponding to all or a portion of a nucleic acid molecule of the invention is by standard synthetic techniques, e.g., using an automated DNA synthesizer.

Construction of genomic DNA libraries, preparation of cDNA libraries, or isolation of total mRNA from the identified source cell, can be performed by standard techniques known in the art. These techniques can be found, for example, in Maniatis et al., Molecular Cloning: A Laboratory Manual, Second Edition (Cold Spring Harbor Laboratory, Cold Spring Harbor, N.Y., 1989). An exemplary method on isolation of the rat GPCR135s nucleic acid molecule is described in Example 1.

In another embodiment, the present invention provides vectors, preferably expression vectors, containing a nucleic acid that is capable of expressing a rat GPCR135s.

The recombinant expression vectors of the invention comprise a nucleic acid of the invention in a form suitable for expression of the nucleic acid in a host cell. Thus, the recombinant expression vectors include one or more regulatory sequences, selected on the basis of the host cells to be used for expression, operably linked to the nucleic acid sequence to be expressed. When used in reference to a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g., Escherichia coli (E. coli)) or eukaryotic cells (e.g., insect cells (using baculovirus expression vectors), yeast cells or mammalian cells). Suitable host cells are known to those skilled in the art. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve four purposes: 1) to increase expression of recombinant protein; 2) to increase the solubility of the recombinant protein; 3) to aid in the purification of the recombinant protein by acting as a ligand in affinity purification; and 4) to facilitate detection of the recombinant protein by serving as a marker. Often in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith et al., (1988), Gene 67:31-40), pMAL (New England Biolabs, Beverly, Mass.), pRIT5 (Pharmacia, Piscataway, N.J.), or pQE (Qiagen), which fuse glutathione S-transferase (GST), maltose binding protein, protein A, or poly-His, respectively, to the target recombinant protein.

Examples of suitable inducible non-fusion E. coli expression vectors include pTrc (Amann et al., (1988), Gene 69:301-315) and pETIId (Studier et al., Gene Expression Technology: Methods in Enzymology 185, Academic Press, San Diego, Calif. (1990) 60-89). One strategy to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari et al. (1987), EMBO J. 6:229-234), pMFa (KurJan et al. (1982), Cell 30:933-943), pJRY88 (Schultz et al. (1987), Gene 54:113-123), pYES2 (Invitrogen Corporation, San Diego, Calif.), and pPicZ or Pichia (Invitrogen Corp, San Diego, Calif.).

Alternatively, the expression vector is a baculovirus expression vector. Baculovirus vectors available for expression of proteins in cultured insect cells include, e.g., the pAc series (Smith et al. (1983), Mol. Cell Biol. 3:2156-2165) and the pVL series (Lucklow et al. (1989), Virology 170:31-39). Commercially available insect cell expression vectors useful for recombinant expression include pBlueBacII (Invitrogen).

In yet another embodiment, the expression vector is a mammalian expression vector. When used in mammalian cells, the expression vector's control functions are often provided by viral regulatory elements. For example, commonly used promoters are derived from polyoma, Adenovirus 2, cytomegalovirus and Simian Virus 40. Examples of mammalian expression vectors include, e.g., pCDM8 (Seed (1987), Nature 329:840) and pMT2PC (Kaufinan et al., (1987), EMBO J. 6:187-195). Commercially available mammalian expression vectors which can be suitable for recombinant protease COX-3 expression include, for example, pMAMneo (Clontech), pcDNA3 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and 1ZD35 (ATCC 37565).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Examples of suitable tissue-specific promoters include the albumin promoter (liver-specific; Pinkert et al. (1987), Genes Dev. 1:268-277), lymphoid-specific promoters (Calame et al. (1988), Adv. Immunol. 43:235-275), in particular promoters of T cell receptors (Winoto et al. (1989), EMBO J. 8:729-733) and immunoglobulins (BaneiJi et al. (1983), Cell 33:729-740; Queen et al., (1983), Cell 33:741-748), neuron-specific promoters (e.g., the neurofilament promoter; Byme et al. (1989), Proc. Natl. Acad. Sci. USA 86:5473-5477), pancreas-specific promoters (Edlund et al. (1985), Science 230:912-916), and mammary gland-specific promoters (e.g., milk whey promoter; U.S. Pat. No. 4,873,316 and European Patent Publication No. 264,166). Developmentally regulated promoters also include, for example, the marine hox promoters (Kessel et al. (1990), Science 249:374-379) and the beta-fetoprotein promoter (Campes et al. (1989), Genes Dev. 3:537-546).

The invention further provides a recombinant vector comprising a DNA molecule of the invention cloned into the vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense-RNA molecule in a variety of cell types, for instance viral promoters and/or enhancers, or regulatory sequences can be chosen which direct constitutive, tissue-specific or cell-type specific expression of antisense RNA. The antisense expression vector can be in the form of a recombinant plasmid, phagemid or attenuated virus in which antisense nucleic acids are produced under the control of a high-efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

The invention also provides a recombinant vector system that directs the synthesis of small interfering RNAs (siRNAs) in mammalian cells. Many organisms possess mechanisms to silence any gene expression when double-stranded RNA (dsRNA) corresponding to the gene is present in the cell through a process known as RNA interference. The technique of using dsRNA to reduce the activity of a specific gene was first developed using the worm C. elegans and has been termed RNA interference or RNAi (Fire et al. (1998), Nature 391: 806-811). RNAi has since been found to be useful in many organisms, and recently has been extended to mammalian cells in culture (see review by Moss (2001), Curr Biol 11: R772-5). An important advance was made when RNAi was shown to involve the generation of small RNAs of 21-25 nucleotides (Hammond et al. (2000), Nature 404: 293-6; Zamore et al., (2000) Cell 101: 25-33). These small interfering RNAs, or siRNAs, may initially be derived from a larger dsRNA that begins the process, and are complementary to the target RNA that is eventually degraded. The siRNAs are themselves double-stranded with short overhangs at each end; they act as guide RNAs, directing a single cleavage of the target in the region of complementarity (Elbashir et al. (2001) Genes Dev 15: 188-200; Zamore et al. (2000) Cell 101: 25-33).

An siRNA comprising 21-25 nucleotides that are complementary to rat GPCR135s as depicted in SEQ ID NO: 11, can be produced in vitro, for example using a method described in WIPO Publication No. WO01/75164 A2, or can be produced in vivo from a mammalian cell using a stable expression system. An exemplary vector system that directs the synthesis of siRNAs in mammalian cells is the pSUPER (Brummelkamp et al. (2002) Science 296: 550-3). On the pSUPER, the H1-RNA promoter was cloned in front of the gene specific targeting sequence (19-nt sequences from the target transcript separated by a short spacer from the reverse complement of the same sequence) and five thymidines (T5) as a termination signal. The resulting transcript is predicted to fold back on itself to form a 19-base pair stem-loop structure, resembling that of C. elegans Let-7. The size of the loop (the short spacer) is preferably 9 bp. A small RNA transcript lacking a poly-adenosine tail, with a well-defined start of transcription and a termination signal consisting of five thymidines in a row (T5) was produced. Notably, the cleavage of the transcript at the termination site is after the second uridine, yielding a transcript resembling the ends of synthetic siRNAs, that also contain two 3′ overhanging T or U nucleotides. The siRNA expressed from PSUPER is able to knock down gene expression as efficiently as the synthetic siRNA.

Vectors of the present invention also include specifically designed vectors that allow the shuttling of DNA between hosts such as bacteria-yeast or bacteria-animal cells or bacteria-fungal cells or bacteria-invertebrate cells. Numerous cloning vectors are known to those skilled in the art and the selection of an appropriate cloning vector is within the purview of the artisan. For other suitable expression systems for both prokaryotic and eukaryotic cells see, e.g., chapters 16 and 17 of Maniatis et al., supra.

The present invention also provides recombinant host cells into which a recombinant vector of the invention has been introduced.

Cell lines derived from mammalian species which can be suitable for transfection and which are commercially available, include, e.g., CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), Drosophila or murine L-cells, and HEK-293 (ATCC CRL1573), and monkey kidney cells.

Vector DNA can be introduced into prokaryotic or eukaryotic cells via conventional transformation or transfection techniques. As used herein, the terms “transformation” or “transfection” refers to a process by which cells take up foreign DNA and may or may not integrate that foreign DNA into their chromosome. Transfection can be accomplished, for example, by various techniques including calcium phosphate or calcium chloride co-precipitation, DEAE-dextran-mediated transfection, lipofection, electroporation, or protoplast fusion. Suitable methods for transforming or transfecting host cells can be found in Maniatis et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

In another embodiment, the expression characteristics of an endogenous nucleic acid within a cell, cell line or microorganism can be modified by inserting a DNA regulatory element heterologous to the endogenous gene of interest into the genome of a cell, a stable cell line or a cloned microorganism such that the inserted regulatory element is operatively linked with the endogenous gene and controls, modulates or activates the endogenous gene.

A heterologous regulatory element can be inserted into a stable cell line or cloned microorganism, such that it is operatively linked with and activates expression of endogenous genes, using techniques such as targeted homologous recombination, e.g., as described in U.S. Pat. No. 5,272,071 and WIPO Publication No. WO 91/06667.

Another embodiment of the invention provides a substantially purified polypeptide consisting essentially of the amino acid sequence of SEQ ID NO: 15.

The isolated polypeptide of the invention also includes chimeric or fusion proteins comprising a polypeptide of SEQ ID NO: 15 operably linked to a heterologous polypeptide, wherein the heterologous polypeptide is not naturally associated with a rat GPCR135s isoform. For example, the first seven amino-acid sequence at the N-terminal of the longer isoform of rat GPCR135 is excluded from being the heterologous polypeptide because it is naturally associated with the longer isoform of rat GPCR135. When used in reference to a fusion protein, the term “operably linked” is intended to indicate that the polypeptide and the heterologous polypeptide are fused in-frame to each other. The heterologous polypeptide can be fused to the N-terminus or C-terminus of the polypeptide.

One example of a useful fusion protein is an HAHis fusion protein in which the polypeptide of the invention is fused at the C-terminus to a tag made of HA and poly His. Such fusion proteins facilitate the detection and purification of a recombinant polypeptide of the invention.

The invention also pertains to methods of expressing or isolating the inventive polypeptide. In one embodiment, the polypeptide can be isolated from cells or tissue sources that express it naturally by an appropriate purification scheme using standard protein purification techniques. In another embodiment, polypeptides of the invention are produced by recombinant DNA techniques. Alternatively, a polypeptide of the invention can be synthesized in an in vitro translation and/or transcription system. Further alternatively, a polypeptide of the invention can be synthesized chemically using standard peptide synthesis techniques.

Polypeptides of the invention can be recombinantly expressed by cloning DNA molecules of the invention into an expression vector described above, introducing such a vector into prokaryotic or eukaryotic host cells as described herein, and growing the host cell under conditions suitable for production of recombinant protein. The expression vector-containing cells are clonally propagated and individually analyzed to determine whether they produce the polypeptide of the invention. Identification of the rat shorter isoform GPCR135 expressing host cell clones can be done by several means, including immunological reactivity with anti-rat GPCR135 antibodies, and the presence of host cell-associated GPCR135 activity, such as relaxin3 activated changes in intracellular Ca²⁺ concentration. The selection of the appropriate growth conditions and recovery methods are within the skill of the art. Techniques for recombinantly expressing a polypeptide are described in, e.g., Maniatis et al., supra, and are known in the art.

Polypeptides of the invention can also be produced using an in vitro translation and/or transcription system. Such methods are known to those skilled in the art. For example, synthetic rat GPCR135s mRNA or mRNA isolated from rat GPCR135 shorter isoform producing cells can be efficiently translated in various cell-free systems, including wheat germ extracts and reticulocyte extracts. Alternatively, the coding sequence of rat GPCR135s cDNA can be cloned under the control of a T7 promoter. Then, using this construct as the template, rat GPCR135 shorter isoform protein can be produced in an in vitro transcription and translation system, for example using a TNT T7 coupled Reticulocyte Lysate System such as that commercially available from Promega-(Madison, Wis.).

Polypeptides of the invention can also be produced by chemical synthesis, using methods as described supra.

The rat GPCR135s protein can be purified by methods known to those skilled in the art. For example, it can be purified from cell lysates and extracts from natural or recombinant host cells, by various combinations or individual application of salt fractionation, ion exchange chromatography, size exclusion chromatography, hydroxylapatite adsorption chromatography and hydrophobic interaction chromatography, lectin chromatography, HPLC, and FPLC, and antibody/ligand affinity chromatography.

Production of A Relaxin3 from A Recombinant Cell

Because relaxin3 is a secreted protein composed of two chains (A-chain and B-chain) of polypeptides linked by disulfide bridges, proteases are involved in the production of mature relaxin3 from its pre-propeptide. First, signal sequence peptidase cleaves off the signal sequence from the propeptide, resulting in relaxin3 propeptide. The propeptide is subsequently cleaved into chains A, B, and C of the relaxin3 by pro-hormone convertases. A-chain and B-chain form the final mature relaxin3, and the C-chain is not part of the mature relaxin3. Because pro-hormone convertases are only selectively expressed in certain limited cell types where hormones are secreted, such as some neuron cells, or endocrine cells, the relaxin3 propeptide is often not efficiently processed when it is recombinantly expressed from other cell type, such as a COS7 (Example 4).

The present invention provides a method for production of a relaxin3 from a recombinant cell, comprising the steps of: 1) constructing a DNA molecule capable of encoding a modified relaxin3 propeptide with a protease cleavage site inserted at the peptide junction between chains A and C, and/or chains C and B of the relaxin3 propeptide; 2) constructing a vector capable of expressing the modified relaxin3 propeptide; 3) constructing another vector capable of expressing a protease that can cleave the modified relaxin3 propeptide at the inserted protease cleavage site(s); 4) introducing both the vectors into a host cell; and 5) growing the host cell under conditions suitable for the expression of both the modified relaxin3 propeptide and the protease, so that the protease will efficiently cleave the peptide linkage between chains A and C, or chains C and B on the modified relaxin3 propeptide.

Sequence analyses suggested that furin, a member of the pro-hormone convertases, cleaves off the peptide linkage between chains C and B on the native relaxin3 propeptide, and another unidentified protease cleaves off the peptide linkage between chains A and C on the native propeptide. In a preferred embodiment of the invention, a furin cleavage site, with the amino acid sequence of arg-gly-arg-arg (RGRR), is inserted at the peptide junction of chains A and C. An expression vector for this mutant relaxin3 and another expression vector for furin are co-transfected into a host cell. Under suitable growth conditions, the relaxin3 pro-peptide was almost completely processed into mature peptides, A, B, and C. Note that the insertion of a furin cleavage site at the peptide junction of chains A and C introduced no amino acid changes in chains A and B, which constitute the mature relaxin3. The efficiency of pro-peptide cleavage has significantly increased (more than 90% of the pro-peptide has been cleaved into mature form) as compared with recombinant expression of relaxin3 without the insertion of the furin cleavage site between chains A and C (less than 20% of the pro-peptide has been cleaved into mature form) (see Example 4 below).

Instead of furin, other proteases can also be used in the method of the invention. Preferably, identical protease cleavage sites can be inserted between chains A and C, and chains C and B, on the pro-peptide. Examples of proteases that can be used include, e.g., a pro-hormone convertase, such as Furin, PC1, and PC2 (Hosaka et. al., 1991, J. Biol. Chem. 266:12127-12130; Benjannet et. al., 1991, Proc. Natl. Acad. Sci. USA, 88:3564-3568; Thomas et. al., 1991, Proc. Natl. Acad. Sci. USA, 88:5297-5301).

The present invention also relates to an expression vector that is capable of expressing a modified relaxin3 propeptide with a protease cleavage site inserted at the peptide junction between chains A and C, and/or chains C and B on the relaxin3 propeptide. The invention also relates to a host cell comprising both the expression vector for the modified relaxin3 described above and an expression vector for a protease that recognizes the protease cleavage site incorporated onto the modified relaxin3 propeptide.

In one preferred embodiment, a tag, such as a HA, poly His, or FLAG, can be added to the modified relaxin3 propeptide to facilitate protein isolation or purification. Methods are known to those skilled in the art to modify a DNA molecule to cause certain desirable changes in the amino acid sequence encoded by such DNA molecule. Example 4 illustrates one of such methods, the fusion PCR technique.

The identified interaction between GPCR135 and relaxin3 allows for development of transgenic animals, such as knock-out mice, in which a GPCR135 gene and/or relaxin3 gene has been introduced or disrupted. The identified interaction between the receptor and ligand also allows for the employment of GPCR135, or a GPCR135/relaxin 3 complex, in screening methods or assays for identifying compounds for their potential efficacy in treating a disorder related to the relaxin3 and GPCR135 complex.

Method of Identifying Modulators of the Activity of the Relaxin3/GPCR135 Complex

Thus, another general aspect of the invention relates to a method of identifying modulators that either increase or decrease biological activity of the relaxin3/GPCR135 complex. Such modulators are useful as therapeutic agents in treating a subject suffering from a disease or disorder related to the relaxin3/GPCR135 complex, such as CNS disorders (anxiety, schizophrenia, depression, mood, sleep/wake), metabolic disorders, feeding/drinking disorders, water and nutrient homeostasis, and endocrine disorders (see Goto et al., 2001, J. Comp. Neurol. 438: 86-122).

“Inhibitors” refer to compounds that decrease, prevent, inactivate, desensitize or down-regulate relaxin3/GPCR135 complex expression or activity. “Activators” are compounds that increase, activate, facilitate, sensitize or up-regulate relaxin3/GPCR135 complex expression or activity. “Modulators” include both “inhibitors” and “activators”.

The compound identification methods can be performed using conventional laboratory formats or in assays adapted for high throughput. The term “high throughput” refers to an assay design that allows easy screening of multiple samples simultaneously, and can include the capacity for robotic manipulation. Another desired feature of high throughput assays is an assay design that is optimized to reduce reagent usage, or minimize the number of manipulations in order to achieve the analysis desired. Examples of assay formats include 96-well or 384-well plates, levitating droplets, and “lab on a chip” microchannel chips used for liquid-handling experiments. As known by those in the art, as miniaturization of plastic molds and liquid-handling devices are advanced, or as improved assay devices are designed, greater numbers of samples will be able to be screened more efficiently using the inventive assay.

Candidate compounds for screening can be selected from numerous chemical classes, preferably from classes of organic compounds. Although candidate compounds can be macromolecules, preferably the candidate compounds are small-molecule organic compounds, i.e., those having a molecular weight of greater than 50 and less than 2500. Candidate compounds have one or more functional chemical groups necessary for structural interactions with polypeptides. Preferred candidate compounds have at least an amine, carbonyl, hydroxyl or carboxyl group, preferably at least two such functional groups, and more preferably at least three such functional groups. The candidate compounds can comprise cyclic carbon or heterocyclic structural moieties and/or aromatic or polyaromatic structural moieties substituted with one or more of the above-exemplified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound is preferably a DNA or RNA molecule, although modified nucleic acids having non-natural bonds or subunits are also contemplated.

Candidate compounds may be obtained from a variety of sources, including libraries of synthetic or natural compounds. For example, numerous means are available for random and directed synthesis of a variety of organic compounds and biomolecules, including expression of randomized oligonucleotides, synthetic organic combinatorial libraries, phage display libraries of random peptides, and the like. Candidate compounds can also be obtained using any of the numerous approaches in combinatorial library methods known in the art, including: biological libraries; spatially addressable parallel solid-phase or solution-phase libraries: synthetic library methods requiring deconvolution; the “one-bead one-compound” library method; and synthetic library methods using affinity chromatography selection (see, e.g., Lam (1997), Anticancer Drug Des. 12:145). Alternatively, libraries of natural compounds in the form of bacterial, fungal, plant and animal extracts are available or may be routinely produced. Additionally, natural and synthetically produced libraries and compounds can be routinely modified through conventional chemical, physical, and biochemical means.

Further, known pharmacological agents can be subjected to directed or random chemical modifications, such as acylation, alkylation, esterification, amidification, etc., to produce structural analogs of the agents. Candidate compounds can be selected randomly or can be based on existing compounds that bind to and/or modulate the function of GPCR activity. Therefore, a source of candidate agents is libraries of molecules based on known activators or inhibitors for GPCRs with similar structures to GPCR135, in which the structure of the compound is changed at one or more positions of the molecule to contain more or fewer chemical moieties or different chemical moieties. The structural changes made to the molecules in creating the libraries of analog activators/inhibitors can be directed, random, or a combination of both directed and random substitutions and/or additions.

A variety of other reagents also can be included in the mixture. These include reagents such as salts, buffers, neutral proteins (e.g., albumin), and detergents that can be used to facilitate optimal protein-protein and/or protein-nucleic acid binding. Such a reagent can also reduce non-specific or background interactions of the reaction components. Other reagents that improve the efficiency of the assay, such as nuclease inhibitors, antimicrobial agents, and the like, can also be used.

Examples of methods for the synthesis of molecular libraries can be found in the art, for example in: Zuckermann et al. (1994), J. Med. Chem. 37:2678. Libraries of compounds can be presented in solution (e.g., Houghten (1992), Biotechniques 13:412-421), or on beads (Lam (1991), Nature 354:82-84), chips (Fodor (1993), Nature 364:555-556), bacteria (U.S. Pat. No. 5,223,409), spores (U.S. Pat. No. 5,571,698), plasmids (Cull et al. (1992), Proc. Natl. Acad. Sci. USA 89:1865-1869) or phage (see e.g., Scott and Smith (1990), Science 249:386-390).

Thus, in one general aspect the invention relates to a method of identifying a compound that increases or decreases a biological activity of a relaxin3/GPCR135 complex, comprising the steps of:
(a) contacting a solution comprising a buffer and a candidate or test compound with a assay reagent comprising the relaxin3 and GPCR135 complex;
(b) measuring the biological activity of the relaxin3 and GPCR135 complex; and
(c) comparing the result of step (b) with that of a control wherein the relaxin3 and GPCR135 complex was contacted with only the buffer.

As described above, the relaxin3 and GPCR135 complex comprises a relaxin3 or an active fragment of relaxin3 and a GPCR135 or an active fragment of GPCR135. In a preferred embodiment, the relaxin3 and GPCR135 complex in the method is associated with a cell expressing the GPCR135 on the cell surface.

The term “cell” refers to at least one cell or a plurality of cells appropriate for the sensitivity of the detection method. Cells suitable for the present invention may be bacterial, but are preferably eukaryotic, such as yeast, insect, or mammalian. The cell can be a natural host cell for an endogenous GPCR135, preferably a recombinant host cell for a GPCR135, which expresses increased amount of a mammalian GPCR135 on the cell surface.

The relaxin3 and GPCR135 complex can be formed by adding a relaxin3 to the GPCR135 host cell in the form of a purified protein, or in the form of a cell or tissue extract containing the relaxin3. The relaxin3 can be the full-length mature polypeptide, or a fragment that is still capable of binding to a GPCR135.

In a preferred embodiment, the biological activity of a relaxin3 and GPCR135 complex can be measured by a second messenger response of the cell. For example, the biological activity of the relaxin3 and GPCR135 complex can be measured by the signal transduction event triggered by activated GPCR135. This signal transduction event can be measured indirectly by means of measuring one or more changes in cellular physiology, such as cell morphology, migration, or chemotaxis, using one or more suitable methods known to those skilled in the art. It can also be measured directly by measuring phosphorylation of proteins involved in the signal transduction pathway, for example, the phosphorylation of a GTP-binding protein (G protein). Methods are known to those skilled in the art to measure protein phosphorylation, for example, by using an ATP or GTP molecule that has been radiolabeled on the γ-phosphate.

The biological activity of the relaxin3 and GPCR135 complex can also be measured by the intracellular concentration of a second messenger molecule using any of a number of suitable techniques known to those skilled in the art. For example, the pH change can be measured using a pH sensitive dye, such as Acridine Orange. The calcium concentration can be measured via optical imaging of fluorescent indicators sensitive to Ca²⁺, such as fluo-3 (pentapotassium salt, cell-impermeant form; Molecular Probes) or fluo-3(AM) (an acetoxymethyl ester form of fluo-3, Teflabs) (see for example, Liu et al., 2001, J Pharmacol Exp Ther. 299: 121-30) using a fluorometric imaging plate reader (FLIPR) or a confocal microscope (see Example 6). The cAMP concentration can be detected using a commercially available ELISA kit (FLASHPLATE cyclic AMP assay system (1251), Cat. No: SMP001A, NEN; see also Shimomura et al., 2002, J. Biol. Chem. 277: 35826-32) (see Example 8), or via a reporter system wherein the expression of a reporter gene, such as beta-galactosidase, is under the control of a cAMP responsive element (cre) (Montminy et al., 1990, Trends Neurosci, 13(5): 184-8) (see Example 1).

The test compound can be further characterized by comparing its effect on two cells, the first cell containing a functional GPCR135 and the second one identical to the first, but lacking a functional GPCR135. This technique is also useful in establishing the background noise of these assays. One of ordinary skill in the art will appreciate that this control mechanism also allows ready selection of cellular changes that are responsive to modulation of functional GPCR135. Therefore, in a preferred embodiment, the screening method comprises the steps of: (a) contacting a first cell having a GPCR135 expressed on the cell surface with a relaxin3 or an active fragment of relaxin3 and with a test compound; (b) determining a second messenger response in the first cell to the test compound, and comparing it with that of a control wherein the first cell is only contacted with the relaxin3 or the active fragment thereof but not the test compound; (c) contacting a second cell with a relaxin3 or the active fragment of relaxin3 and with a test compound; wherein the second cell is otherwise identical to the first cell except that it does not express a GPCR135 on the cell surface; (d) determining a second messenger response of the second cell to the test compound, and comparing the second messenger response with that of a control wherein the second cell is only contacted with the relaxin3 but not the test compound; and (e) comparing the comparison result of (b) with that of (d).

There are a number of ways to obtain two cells that are otherwise identical except that one expresses a GPCR135 on its cell surface and the other does not. In one embodiment, the first cell is a recombinant host cell for GPCR135 that constitutively expresses GPCR135 on its cell surface, and the second cell is the parent cell from which the GPCR135 recombinant cell is constructed. In another embodiment, a recombinant host cell for GPCR135 is constructed such that the expression of GPCR135 on the cell surface is under the control of an inducible promoter. The first cell is the recombinant cell grown under inducible conditions that allows the expression of GPCR135 on its cell surface, and the second cell is the recombinant cell grown under non-inducible conditions that do not allow the expression of GPCR135. In yet another embodiment, the first cell is a native host cell for GPCR135 that expresses GPCR135 on its cell surface, and the second cell is a mutant cell derived from the native host, wherein the GPCR135 gene has been inactivated through mutagenesis. Standard molecular biology methods can be used to construct a recombinant host cell for GPCR135, or to inactivate a GPCR135 gene.

In another preferred embodiment, the present invention provides a method of identifying a compound that increases or decreases the activity of a relaxin3/GPCR135 complex, comprising the steps of: (a) contacting an isolated membrane preparation comprising a GPCR135 with a relaxin3 or an active fragment of relaxin3, with a test compound, and with a GTP molecule that has been labeled on the □-phosphate; and (b) determining the amount of labeling bound to the membrane preparation; and (c) comparing the amount of labeling in (b) with that of a control wherein the membrane preparation is only contacted with the relaxin3 or the active fragment thereof and the labeled GTP but not the test compound.

The membrane preparation can be isolated from a native host cell that expresses GPCR135 on its cell surface, or preferably, from a recombinant host cell that expresses increased amount of GPCR135 on its cell surface. It can also be isolated from tissues comprising GPCR135 host cells. Methods of membrane preparation are illustrated in the Examples below.

A variety of labels can be used to label the GTP molecule on the γ-phosphate, such as a fluorescent molecule or a radioactive isotope such as ³⁵S, ³²P, and the like. An exemplary method of using [³⁵S]GTPγS as the labeling molecule is described in Example 5.

In yet another embodiment, the present invention provides a method of identifying a compound that binds to a GPCR135, comprising the steps of: a) contacting a GPCR135 with a test compound, and with a labeled relaxin3 or an active fragment thereto; b) measuring the amount of the labeled relaxin3 or the fragment thereof that binds to the GPCR135; and c) comparing the measured amount of (b) with that of a control, wherein the GPCR135 is only contacted with a labeled relaxin3 or the fragment thereof, but not the test compound.

In one preferred embodiment, a GPCR135 host cell (recombinant or native) that expresses the GPCR315 on the cell surface can be used for the binding assay. In another preferred embodiment, isolated membrane preparations comprising the GPCR135 can be used for the binding assay. In yet another preferred embodiment, a substantially purified extracellular fragment of GPCR135 that is capable of binding to a relaxin3 can be used for the binding assay.

The amount of the labeled relaxin3 or fragment thereof that binds to the GPCR135 can be measured by first separating the unbound labeled relaxin3 or fragment thereof from the GPCR135, and then measuring the amount of labeling that is associated with the GPCR135.

Separation of the GPCR135 protein from unbound labeled relaxin3 or fragments thereof can be accomplished in a variety of ways. Conveniently, the GPCR135 may be immobilized on a solid substrate, from which the unbound relaxin3 can be easily separated. The solid substrate can be made of a variety of materials and in a variety of shapes, e.g., microtiter plate, microbead, dipstick, resin particle, etc. The substrate preferably is chosen to maximize signal-to-noise ratios, primarily to minimize background binding, as well as for ease of separation and cost.

Separation can be effected by, for example, removing a bead or dipstick from a reservoir, emptying or diluting a reservoir such as a microtiter plate well, or rinsing a bead, particle, chromatographic column or filter with a wash solution or solvent. The separation step preferably includes multiple rinses or washes. For example, when the solid substrate is a microtiter plate, the wells can be washed several times with a washing solution, e.g., that includes those components of the incubation mixture that do not participate in specific bindings, such as salts, buffer, detergent, non-specific protein, etc. Where the solid substrate is a magnetic bead, the beads can be washed one or more times with a washing solution and isolated using a magnet.

GPCR135 can be immobilized on a solid substrate using a number of methods. In one embodiment, a fusion protein can be provided which adds a domain that allows the GPCR135 proteins to be bound to a matrix. For example, glutathione-S-transferase fusion proteins or glutathione-S-transferase fusion proteins can be adsorbed onto glutathione sepharose beads (Sigma Chemical, St. Louis, Mo.) or glutathione derivatized microtiter plates, which are then combined with the test compound and the labeled relaxin3, and the mixture is incubated under conditions conducive to complex formation (e.g., at physiological conditions for salt and pH). Following incubation, the beads or microtiter plate wells are washed to remove any unbound components and complex formation is measured either directly or indirectly, for example, as described above. Alternatively, the complexes can be dissociated from the matrix, and the level of binding or the labeled relaxin3 to GPCR135 can be determined using standard techniques.

Other techniques for immobilizing proteins on matrices can also be used in the screening assays of the invention. For example, the GPCR315 can be immobilized utilizing conjugation of biotin and streptavidin. Biotinylated polypeptide can be prepared from biotin-NHS(N-hydroxy-succinimide) using techniques known in the art (e.g., biotinylation kit available from Pierce Chemicals, Rockford, Ill.), and immobilized in the wells of streptavidin-coated 96 well plates (Pierce Chemicals).

Alternatively, antibodies reactive with the GPCR135 but which do not interfere with binding of the GPCR135 to relaxin3 or test compound can be attached to the wells of the plate, and GPCR135 then trapped in the wells by antibody conjugation.

A variety of labels can be used to label the relaxin3 or fragments thereof, such as those that provide direct detection (e.g., radioactivity, luminescence, optical or electron density, etc), or indirect detection (e.g., epitope tag such as the FLAG epitope, enzyme tag such as horseradish peroxidase, etc.).

Interaction of the GPCR135 to relaxin3 in the presence and absence of a candidate compound can be accomplished in any vessel suitable for containing the reactants. Examples of such vessels include microtiter plates, test tubes, and micro-centrifuge tubes.

An exemplary binding assay is described in Example 7 below.

In another general aspect, the invention relates to a method for identifying a compound that binds GPCR135 and mimics relaxin3, comprising the steps of:
(a) contacting a test compound with an assay reagent comprising GPCR135 or an active fragment thereof;
(b) measuring a biological activity of the GPCR135 or active fragment thereof; and
(c) comparing the result of step (b) with that of a control wherein the GPCR135 or an active fragment thereof was contacted with relaxin3 or an active fragment thereof in the absence of the test compound.

In one preferred embodiment to this method, the GPCR135 or active fragment thereof is expressed from a recombinant cell, preferably on the cell surface. In another preferred embodiment, the GPCR135 or active fragment thereof is within an isolated cell membrane preparation.

The biological activity can be any of the biological activities associated with the receptor and ligand complex or the interaction of GPCR135 and relaxin3, such as the signal transduction event or the changes in intracellular concentration of a second messenger molecule triggered by activated GPCR135. These biological activities can be measured using methods described supra. A test compound that “mimics” relaxin3 elicits a similar change in the biological activity of GPCR135 as that of relaxin3.

### EXAMPLE 1

### Cloning And Recombinant Expression of Mammalian GPCR135

This example describes the cloning and recombinant expression of GPCR135 from human, mouse, and rat. Using similar methods, GPCR135 from other animals can also be cloned and recombinantly expressed.

GPCR135 was identified as an orphan GPCR from human genomic DNA (Genbank Accession No.: NT_{—}023085) based on its sequence homology to known GPCRs, the somatostatin receptors. Sequence analyses revealed that the entire coding region of GPCR135 in human, mouse, or rat contains no intron. Therefore, the coding sequence for GPCR135 in these animals can be obtained by PCR amplification using genomic DNA as template, and primers complementary to the putative translation start and stop sites of the GPCR135 open reading frame. Unlike human or mouse, rat has two translation start sites for GPCR135, which are 21 nt apart. The two start sites gave rise to two isoforms of rat GPCR135, the long isoform GPCR1351 and the short isoform GPCR135s, which were cloned and characterized.

Human GPCR135 was PCR amplified from human genomic DNA using the forward primer, SEQ ID NO:1, 5′ ACA GCT CGA GGC CAC CAT GCA GAT GGC CGA TGC AGC CAC G 3′ and the reverse primer, SEQ ID NO:2, 5′ ACA TCA TCT AGA TCA GTA GGC AGA GCT GCT GGG CAG CAG 3′. Mouse GPCR135 coding region was PCR amplified from mouse genomic DNA using the forward primer, SEQ ID NO:3, 5′ ACG ATA CTC GAG GCC ACC ATG CAG GTG GCT TCT GCA ACC CCC GCG 3′, and the reverse primer, SEQ ID NO:4, 5′ ACT AGA TCT AGA TCA GTA GGC AGA GCT ACT AGG GAG CAG GT 3′. Rat GPCR135 long form was PCR amplified from rat genomic DNA, using the forward primer, SEQ ID NO:5, 5′ ACG ATA CTC GAG GCC ACC ATG CCC AAA GCG CAC CTG AGC ATG CAA GT 3′, and the reverse primer, SEQ ID NO:6, 5′ ACG ATA TCT AGA TCA GTA GGC AGA GCT GCT AGG GAG AAG GT 3′. Rat GPCR135 short form was PCR amplified from rat genomic DNA, using the forward primer, SEQ ID NO:7, 5′ ACG ATA CTC GAG GCC ACC ATG CAA GTG GCT TCT GCA ACC ACC GCA 3′, and the reverse primer, SEQ ID NO:6, 5′ ACG ATA TCT AGA TCA GTA GGC AGA GCT GCT AGG GAG AAG GT 3′.

These PCR products were cloned into mammalian expressing vector pCIneo (Promega) between Xho1 and Xba1 sites, separately, and were sequenced to confirm the sequence identities. Sequencing results illustrated that the coding sequence for each of human GPCR135, mouse GPCR135, rat GPCR1351, and rat GPCR135s, is as depicted in SEQ ID NO:8, SEQ ID NO:9, SEQ ID NO:10, and SEQ ID NO:11, respectively. Based on the cDNA sequence, the deduced amino acid sequence for each of human GPCR135, mouse GPCR135, rat GPCR135L, and rat GPCR135S, is as depicted in SEQ ID NO: 12, SEQ ID NO: 13, SEQ ID NO: 14, and SEQ ID NO: 15, respectively.

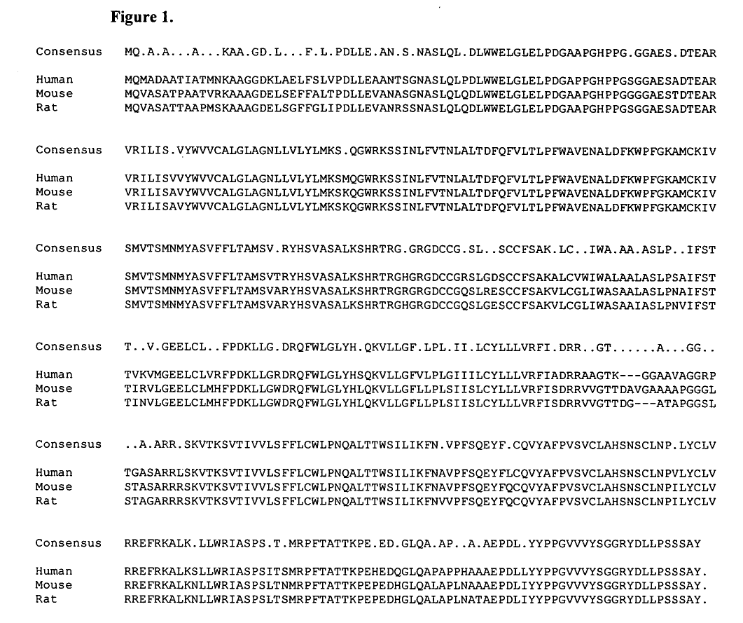

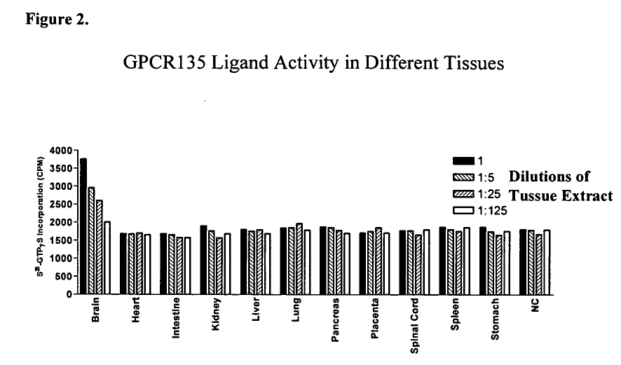

Sequence analysis showed that mammalian GPCR135 shares significant sequence homology to known G-protein coupled receptors such as somatostatin receptor and angiotensin receptor. Amino acid sequence comparison among human, rat and mouse GPCR135 indicated that GPCR135 is a very conserved gene in different animal species (FIG. 1). Rat GPCR1351 has been previously described in WO00/24891. However, sequence comparison indicated that the first amino acid of the rat GPCR135s aligns with the first amino acid of human and mouse GPCR135, suggesting that the rat GPCR135s is likely expressed in rat. It was shown that the recombinantly expressed rat GPCR135s and GPCR1351 have similar activities in radio-ligand binding or GTPγS binding assays (FIG. 2).

To study the tissue expression pattern of the human GPCR135 mRNA, quantitative PCR amplification assays were performed on cDNAs from different human tissues (Clontech) using primers that anneal to human GPCR135 gene. The resulted PCR products were run on a 1% agarose gel and stained with ethdium brimide. As the control, human actin primers were used to PCR amplify human actin cDNA from the same tissue. The results of agarose gel analyses showed that human GPCR135 mRNA is most strongly expressed in brain, with some weak expression in placenta, pancreas, and testis. No or very weak expression was found in other tissues tested including colon, heart, kidney, liver, lung, spleen, skeleton muscles, and thymus. Consistent results on the expression of human GPCR135 mRNA have been obtained from other mRNA expression analysis, such as RT-PCR, RNase protection, and in situ hybridization.

To obtain a recombinant GPCR135 host cell, the above GPCR135 expressing vector was transfected into COS-7 (ATCC# CRL-1651), CHO-K1 (ATCC# CCL-61) cells using LipofectAmine (Invitrogen, Cat No: 18324-020). Two days after transfection, the cells were used for radio-ligand binding assays or GTPγS binding assays (see Example 5). In a separate experiment, the GPCR135 expressing vector was either co-transfected with Gqi5 or by itself into 293 (ATCC# CRL-1573) cell using LipofectAmine. Two days after transfection, the cells were used for Ca²⁺ mobilization assay in response to ligand stimulation using FLIPR (see Example 6). In addition, GPCR135 expressing vector was also transfected into SK-N-MC cells (ATCC# HTB-10) harboring a CRE-β gal reporter gene. The stable cell line was established by culturing the cells in minimum essential medium plus 10% fetal bovine serum with selection of G418 (400 mg/L), and was used in reporter assay for intracellular cAMP concentration. In the absence of the GPCR135 expression vector, none of the COS-7, CHO-K1, HEK293, and SK-N-MC cells expresses GPCR135 at detectable levels from GTPγS binding assays.

### EXAMPLE 2

### Initial Identification and Characterization Of GPCR135 Ligand Activity from Rat Brain

This example describes the identification and characterization of the GPCR135 ligand activity from the rat brain. Using similar methods, the ligand activity can also be studied in other animal brain tissues, such as from human, mouse, dog, and pig.

Frozen rat brains (5 g) were homogenized at −30° C. in 40 ml ethanol/HCl (ethanol to 0.8M HCl ratio at 3:1). The homogenate was extracted at 4° C. for 2 hr and then centrifuged at 4° C. at 20,000 g for 30 min. The supernatant was loaded onto a 2 ml SP-Sephadex C-25 column (Amersham), washed with 1 M acetic acid and eluted with 2 M pyridine and 1 M acetic acid. The eluted fraction was loaded onto a 6 ml C-18 BondElut column, washed with 0.1% TFA and eluted with 60% acetonitrile and 0.1% TFA. The eluted peptides were lyophilized, reconstituted in water, and tested for GPCR135 ligand activity using GTPγS binding assay as described in Example 5.

The molecular size of the rat GPCR135 ligand was characterized by HPLC gel filtration. The reconstituted fraction from the rat brain extract was run through a HPLC gel filtration column (BioSep-SEC-S 2000, Phenomenex) in 1 M acetic acid. Fractions of 1.0 ml were collected during the HPLC separation, lyophilized, and tested for GPCR135 ligand activity using GTPγS binding assays. In a parallel experiment, proteins or peptides with various molecular weights were run in the same to serve as the molecular weight standards.

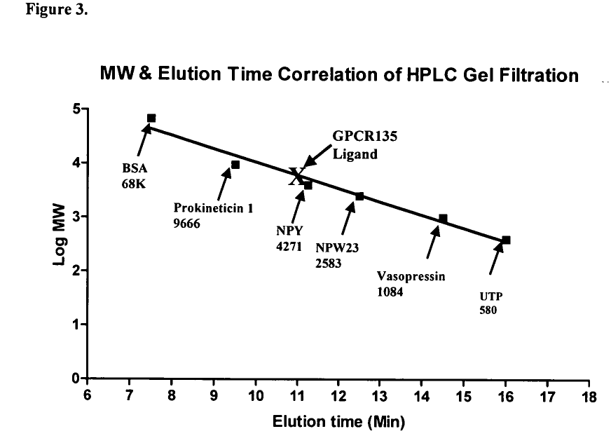

Results showed that rat brain extract potently stimulated the GTPγS binding to isolated membranes containing GPCR135, but not to the negative controls containing no GPCR135. HPLC gel filtration analysis of the rat brain extract indicated that the GPCR135 ligand activity was eluted around MW of 4000-5000 (FIG. 3). Because other types of GPCR ligand, such as lipid, nucleotide, or small molecules, seldom have a molecular weight (MW) exceeding 4000, the molecular weight study suggested that the GPCR135 ligand is a peptide.

### EXAMPLE 3

### Purification of GPCR135 Ligand from Porcine Brain

To further characterize the GPCR135 ligand, it can be purified from a brain extract. This example describes the purification of the GPCR135 ligand from porcine brain. Similar methods can be used to purify the ligand from other animals.

Frozen porcine brains (5 kg) were homogenized in cold ethanol/HCl as described above for rat brain extraction with increased scale. The supernatant of porcine brain extract was loaded onto a resource S cation ion exchange column and eluted with 20 mM sodium acetate buffer (pH 5.0) and 20% acetonitrile with NaCl gradient from 0 to 2 M. Fractions that stimulated the GTPγS binding in GPCR135 expressing cell membrane were pooled and loaded on resource S cation ion exchange column at pH 2.5 and eluted with 20 mM sodium phosphate buffer (pH 2.5) and 20% acetonitrile with gradient of NaCl from 0 to 2 M. The active fractions were pooled and loaded onto an RP-HPLC semi-prep column and eluted with 0.1% TFA/acetonitrile gradient. The active fractions were pooled and lyophilized, re-dissolved in 0.1% TFA and then loaded onto an analytical RP-HPLC column. The peptides bound to the column were eluted again with a 0.1% TFA/acetonitrile gradient. At this stage the active fraction appeared to be pure. The peptide in the fraction had a MW of 5500 as indicated by mass spectrometry analyses, and the N-terminus of the peptide was sequenced using Edman degradation method. It was found that the purified fraction consisted of two peptides with the N-terminal sequences as depicted in SEQ ID NO:16, DVLAGLSSNXXKWGXSKSEI, and SEQ ID NO:17, RASPYGVKLXGREFIRAVIF.

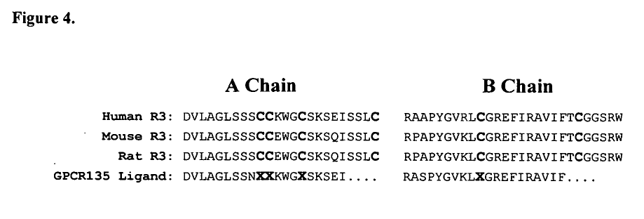

Database searching with the derived sequences as queries revealed that both the sequences match the sequence of relaxin3, also referred to as insulin-like peptide 7 (Accession No. NM_{—}173184, mouse) (FIG. 4), an insulin family member that contains two peptides linked by disulfide bonds. This result indicated that the purified and sequenced peptide is porcine relaxin3, supporting that relaxin3 is the ligand for GPCR135.

### EXAMPLE 4

### Expression And Purification Of Human Recombinant Relaxin3

The complete coding sequence of human relaxin3 was PCR amplified from human brain cDNA library (Clontech) using the forward primer, SEQ ID NO:18, 5′ ACG ATC GTC GAC GCC ACC ATG GCC AGG TAC ATG CTG CTG CTG CTC 3′, and the reverse primer, SEQ ID NO:19, 5′ ACG ATA AAG CTT CTA GCA AAG GCT ACT GAT TTC ACT TTT GC 3′. The PCR product was cloned into a mammalian expression vector pCMV-sport1 (Invitrogen) between Sal1 and BamH1 sites. The cloned cDNA was sequenced to confirm the identity.

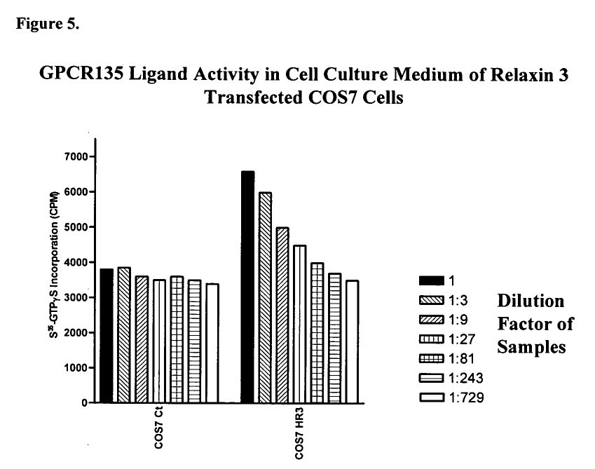

The expression vector was transfected into COS-7 cells using LipofectAmine (Invitrogen). Three days after the transfection, the supernatants of the transfected cells were collected, the pH was adjusted to 3.0 and loaded onto a Sephadex C-25 cation exchange column. The column was washed with 1 M acetic acid and eluted with 2 M pyridine and 1 M acetic acid. The eluted proteins were loaded on a C-18 BondElut column, washed with 0.1% TFA, and eluted with 60% acetonitrile and 0.1% TFA. The eluted proteins were lyophilized, reconstituted in 50 mM Tris-HCl, pH 7.5, and tested in GTPγS binding assays. The results showed that medium from human relaxin3 transfected cell culture potently stimulated the GTPγS binding in GPCR135 expressing cell membrane while the control medium had no activity (FIG. 5), indicating that relaxin3 is indeed the ligand for GPCR135.

To facilitate the purification of the relaxin3 peptide, an expression vector was constructed that encodes a secreted fusion protein comprising the relaxin3 peptide and a FLAG tag at the N-terminus of the relaxin3. Such an expression vector is constructed by replacing the signal sequence of relaxin3 with an alpha peptide signal sequence followed by a FLAG sequence.

The pro-peptide coding region of human relaxin3 was PCR amplified using the forward primer, SEQ ID NO:20, 5′ ACG ATA GAA TTC GAT GAC GAC GAT AAG CGG GCA GCG CCT TAC GGG GTC AGG C 3′, and the reverse primer, SEQ ID NO:21, 5′ ACT ATA GGA TCC CTA GCA AAG GCT ACT GAT TTC ACT TTT GCT AC 3′ with human relaxin3 cDNA as template. The PCR products were cloned into a modified pCMV-sport1 vector, where the poly-cloning sites were modified by replacing the sequence between the Pst1 and EcoR1 sites with a sequence that codes an alpha peptide signal sequence followed by a FLAG tag, SEQ ID NO:22, 5′ CTG CAG GCC GCC ATG CTG ACC GCA GCG TTG CTG AGC TGT GCC CTG CTG CTG GCA CTG CCT GCC ACG CGA GGA GAC TAC AAG GAC GAC GAT GAC AAG GAA TTC 3′. The human relaxin3 pro-peptide coding region was then cloned downstream of FLAG tag between EcoR1 and BamH1 sites. The resulting clone was sequenced to confirm the identity.

The FLAG-relaxin3 fusion peptide expressing plasmid was transfected into COS-7 cells. Two days after transfection, the recombinant fusion peptide was affinity purified from the cell culture medium using an anti-FLAG affinity gel (Sigma). Briefly, the cultured medium was loaded onto the anti-FLAG affinity column. The column was washed with a phosphate buffered saline (PBS) solution, and eluted with 0.1 M Glycine-HCl, pH 2.8. The eluted protein was neutralized with 1 M Tris-HCl, pH 7.5. The N-terminal FLAG tag was cleaved from the fusion peptide by enterokinase (Novagen), and the wild type relaxin3 peptide was further purified by reverse phase HPLC using a C-18 column and a 0.1% TFA/actetonitrile gradient and tested in a GTPγS binding assay. The results showed that the purified peptide potently stimulated GTPγS binding in GPCR135 expressing cell membrane. However, SDS-PAGE and reversed-phase HPLC showed that the purified peptide was heterogeneous, suggesting the C peptide processing was not complete.

To increase the processing efficiency of the pro-relaxin3 into mature relaxin3 by pro-hormone convertase, a furin cleavage site consisting of amino acid sequence RGRR was created in pro-relaxin3 at the junction of the C-chain and A chain. This mutation was created by a two-step overlapping PCR reaction. The 5′ end was PCR amplified using the forward primer (P1), SEQ ID NO:23, ACG ATA CTG CAG GCC GCC ATG CTG ACC GCA GCG TTG CTG A 3′, and the reverse primer (P2), SEQ ID NO:24, 5′ CAG CCA GGA CAT CTC GTC GGC CCC GAA GAA CCC CAG GGG TTC CTT G 3′, with the FLAG-relaxin3 cDNA as the template. The 3′ end was PCR amplified using the forward primer (P3), SEQ ID NO:25, 5′ GGT TCT TCG GGG CCG ACG AGA TGT CCT GGC TGG CCT TTC CAG CAG C 3′, and the reverse primer (P4), SEQ ID NO:26, 5′ ACT ATA GGA TCC CTA GCA AAG GCT ACT GAT TTC ACT TTT GCT AC 3′, with the FLAG-relaxin3 cDNA as the template. The 5′ end and the 3′ end PCR products were purified and mixed together as the template for the second step PCR using forward primer (P1) and the reverse primer (P4) as described above. The final PCR product was cloned into pCMV-sport1 between Pst1 and BamH1 sites, and the insert region was sequenced to confirm the identity.

The new relaxin3 expression vector was co-transfected with a human pro-hormone convertase furin expressing vector into COS-7 cells using LipofectAmine as the transfection reagent. The human furin expressing vector was constructed by PCR amplifying human furin cDNA from human cDNA library using two primers with the forward primer, SEQ ID NO:27, 5′ GAC TAG AAG CTT GCC ACC ATG GAG CTG AGG CCC TGG TTG CTA TG 3′ and the reverse primer, SEQ ID NO:28, 5′ GAC GAT AGC GGC CGC AGT GGG CTC ATC AGA GGG CGC TCT G 3′. The PCR product was cloned into pcDNA3.1/zeo (Invitrogen) between Hind III and Not1 sites. The insert region of the furin expressing vector was sequenced to confirm its identity. The secreted relaxin3 was then purified using an anti-FLAG affinity column, cleaved with enterokinase, and subjected to reversed phase HPLC as described above. Purified relaxin3 from the transfected cell culture medium again potently stimulated GTPγS binding in GPCR135 expressing cell membrane. SDS-PAGE showed a single band of the purified protein. HPLC analysis indicated that the relaxin3 pro-hormone was completely processed into mature peptide and uniform in retention time.

### EXAMPLE 5

### GTPγS Binding Assays

The activity of a GPCR135 ligand (or test compound) can be assayed by a number of procedures measuring the activity of a ligand/GPCR receptor complex or interaction. An example of such an assay is the GTPγS binding assay.

GPCR135 expression vector was transfected into CHO-K1 cells using Lipofectamine (Invitrogen). Two days after transfection, the transfected cells were harvested and homogenized in GTPγS binding buffer: 50 mM Tris-HCl, pH 7.4, 10 mM MgCl2, 1 mM EDTA pH 8.0, and 100 mM NaCl. Protease inhibitors were added to the buffer at concentrations of 1 mM PMSF, 10 μg/ml of pepstain A, 10 μg/ml of leupeptin. Cell membranes were isolated by centrifugation at 20,000 g for 30 min. The isolated cell membrane and different concentrations of ligands were added to 96-well plates and incubated at room temperature for 20 min. ³⁵S-GTPγS (NEN) diluted in GTPγS binding buffer was then added to each well at a final concentration of 200 pM in a final volume of 200 □l. The reactions were then allowed to proceed at room temperature for 1 hr, filtered through a 96-well GFC filter plate (Packard) and washed with cold washing buffer: 50 mM Tris-HCl, pH 7.4, 10 mM MgCl2. Microscint-40 (Packard) was added to each well and the plate was counted on a top counter (TopCount NTX, Packard).

FIG. 5 shows the results of a GTPγS binding assay using human GPCR135 expressing cell membrane and the culture medium from the relaxin3 recombinant cells. Increased incorporation of ³⁵S-GTPγS to GPCR135 expressing cell membranes indicated GPCR135 ligand binding activity in the culture medium from the relaxin3 recombinant cells.

### EXAMPLE 6

### Calcium Mobilization Assays

This example describes another procedure that can be used to assay for the activity of a ligand/GPCR receptor complex.

Using LipofectAmine, human embryonic kidney (HEK) 293 cells were co-transfected with a plasmid expressing GPCR135 receptor and a plasmid expressing Gqi5, a mutant G-protein that translates the activation of GPCR135 into an increased intracellular Ca 2+, or increased Ca²⁺ mobilization (Conklin et al., 1993, Nature 363: 274-276). Two days after transfection, cells were detached with PBS plus 10 mM EDTA, pH 8.0. The detached cells were washed with DMEM-F12 medium (without phenol red, Invitrogen) and seeded in black poly-D lysine coated 96-well plates at a cell density of 50,000 cell/well. Calcium dye, Fluo-3 (AM) (TEFLABS, Austin, Tex.) was loaded into the cells and ligand stimulated calcium mobilization was monitored using FLIPR (Molecular Device).

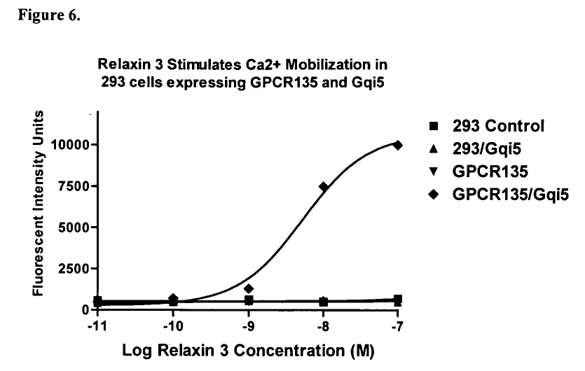

HEK293 cells, untransfected (293 Control) or transfected by Gqi5 (293/Gqi5) alone or human GPCR135 (GPCR135) alone, or co-transfected by human GPCR135 and Gqi5 (GPCR135/Gqi5), were used for Ca²⁺ mobilization assays under stimulation of relaxin3 as the ligand. Ligand-stimulated intracellular mobilization was monitored using FLIPR. As shown in FIG. 6, relaxin3 stimulated Ca²⁺ mobilization in HEK293 cells co-expressing GPCR135 and Gqi5, indicating that relaxin3 binds to and activates GPCR135.

### EXAMPLE 7

### Radioligand Binding Assays

This example describes yet another procedure that can be used to assay for the activity of a ligand/GPCR receptor complex.

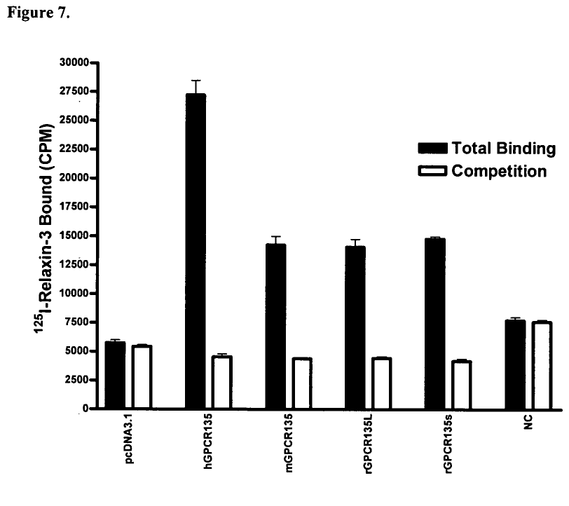

COS-7 cells in 6-well tissue culture plates were transfected by GPCR135 expressing plasmid using LipofectAmine. Two days after transfection, cells were washed with binding buffer: DMEM plus 50 mM Hepes, pH 7.2 and 1% BSA. ¹²⁵I-label human relaxin3 was added to each well in 1 ml of binding buffer at presence or absence 1 μM of unlabeled relaxin as competitor. The binding assays were carried out at room temperature for one hour and the unbound ligand was washed away by ice-cold PBS. The bound 125I-relaxin3 was counted in a gamma counter. As shown in FIG. 7, ¹²⁵I-relaxin 3 specifically binds GPCR135 from either human, mouse, or rat.

### EXAMPLE 8

### Intracellular cAMP Accumulation Assay

This example describes yet another procedure that can be used to assay for the activity of a ligand/GPCR receptor complex.

CHO-K1 cells stably expressing human GPCR135 were established by transfection of GPCR135 expressing plasmid into CHO-K1 (ATCC# CCL-61) cells and culturing the transfected cells under selection of G418 (400 mg/L). The receptor expressing cells were seeded in 96-well plates at a cell density of 30,000 cells/well. Twenty-four hours later, cell culture medium was replaced with DMEM-F12 plus 2 mM IBMX (sigma). A different test compound for cAMP stimulation was added to each well. The reactions were incubated at room temperature for 20 min. Twenty micro-liters of 0.5 N HCl was added to each well to stop the reaction and extract the accumulated cAMP. The cAMP concentration was measured using a cAMP ELISA kit (NEN).

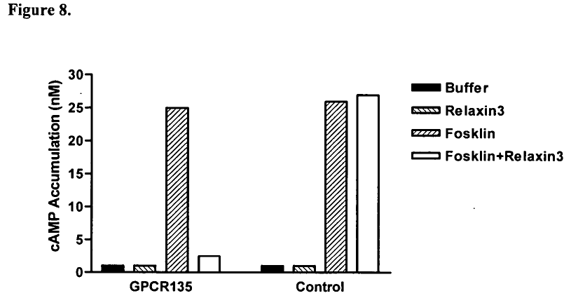

As shown in FIG. 8, forskalin, an agent that is known to stimulate the catalytic activity of adenyl cyclase, increases the intracellular concentration of cAMP in the presence or absence of GPCR135. Relaxin3 activates GPCR135, which in turn inhibits the cyclase activity and results in decreased intracellular accumulation of cAMP. Therefore, relaxin3 inhibits forskalin-stimulated cAMP accumulation in cells expressing GPCR135, but not in control cells where GPCR135 is absent.

Although the various aspects of the invention have been illustrated above by reference to examples and preferred embodiments, it will be appreciated that the scope of the invention is defined not by the foregoing description, but by the following claims properly construed under principles of patent law.

1. A receptor-ligand complex comprising a receptor component containing GPCR135 or an active fragment of GPCR135 bound to a ligand component containing relaxin3 or an active fragment of relaxin3, wherein at least one of the receptor and ligand components is in a substantially pure form.

2. The receptor-ligand complex of claim 1, wherein the receptor component is originated from a human, a mouse, or a rat, and the ligand component is originated from a human, a mouse, or a rat.

3. The receptor-ligand complex of claim 1, wherein said ligand component bears an ¹²⁵I radioisotope label.

4. The receptor-ligand complex of claim 1, wherein the GPCR135 has an amino acid sequence selected from SEQ ID NO: 12, SEQ ID NO: 13, and SEQ ID NO: 15.

5. The receptor-ligand complex of claim 1, wherein said GPCR135 component is a product of expression on the cell surface of a recombinant GPCR135 host cell.

6. The receptor-ligand complex of claim 1, wherein said receptor component contains the GPCR135 or active fragment of GPCR135 associated with isolated cell membranes or lipid vesicles.

7. The receptor-ligand complex of claim 1, wherein both said receptor component and said ligand component are in a substantially pure form.

8. The receptor-ligand complex of claim 1, wherein said ligand component is in a substantially pure form as a product of recombinant expression.

9. The receptor-ligand complex of claim 1, wherein said at least one of the receptor and ligand components is in a substantially pure form as a product of isolation, peptide synthesis, or recombinant expression.

10. The receptor-ligand complex of claim 1, wherein said ligand component is in a substantially pure form as a product of peptide synthesis.

11. An isolated polynucleotide having a nucleotide sequence encoding an amino acid sequence of SEQ ID NO: 15, or a complement thereof.

12. An isolated polypeptide having an amino acid sequence of SEQ ID NO: 15.

13. A vector consisting of a polynucleotide encoding a polypeptide having an amino acid sequence of SEQ ID NO: 15.

14. A recombinant host cell comprising a vector comprising a polynucleotide encoding a polypeptide having an amino acid sequence of SEQ ID NO: 15.

15. A process of producing a relaxin3 from a recombinant cell, comprising:
(a) constructing a DNA molecule encoding a relaxin3 propeptide modified by insertion of a peptide linkage forming a protease cleavage site at at least one location selected from the peptide junction between chains A and C and the peptide junction between chains C and B of the relaxin3 propeptide;
(b) constructing a first vector expressing the modified relaxin3 propeptide;
(c) constructing a second vector expressing a protease for cleaving the modified relaxin3 propeptide at the inserted protease cleavage site;
(d) introducing both the first vector and the second vector into a host cell; and
(e) growing the host cell so that both the modified relaxin3 propeptide and the protease are expressed, whereby the protease efficiently cleaves the peptide linkage at said location on the modified relaxin3 propeptide.

16. The process of claim 15, wherein said modified relaxin3 propeptide has a pro-hormone convertase inserted at said at least one location.

17. The process of claim 15, wherein said modified relaxin3 propeptide has furin inserted at the peptide junction between chains A and C of the relaxin3 propeptide.

18. A method of identifying a compound that increases or decreases a biological activity of a GPCR135/relaxin3 complex, comprising the steps of:
(a) contacting a test sample comprising a compound and a buffering solution with an assay reagent comprising a receptor-ligand complex as defined in claim 1;
(b) determining the biological activity of the receptor-ligand complex; and
(c) comparing the result determined in step (b) with a control measurement wherein the receptor-ligand complex has been contacted with the buffering solution.

19. The method of claim 18, wherein the GPCR135 component of the receptor-ligand complex is a product of expression on the cell surface of a recombinant GPCR135 host cell.

20. The method of claim 19, wherein said determining the biological activity of the receptor-ligand complex comprises measuring a second messenger response.

21. The method of claim 20, wherein said second messenger response is measured by intracellular pH, intracellular calcium ion concentration, or intracellular cAMP concentration.

22. The method of claim 19, wherein the assay reagent comprises an isolated membrane preparation containing the GPCR135 or an active fragment thereof.

23. The method of claim 22, wherein said determining the biological activity of the receptor-ligand complex comprises measuring the amount of protein phosphorylation of the isolated membrane preparation.

24. The method of claim 23, wherein the amount of protein phosphorylation of the isolated membrane preparation is measured using a γ-phosphate labeled GTP molecule.

25. The method of claim 24, wherein the γ-phosphate labeled GTP molecule is selected from ³⁵S-GTPγS, ³³P-GTPγP, and ³²P-GTPγP.

26. A method of identifying a compound that binds to GPCR135 or an active fragment thereof, comprising the steps of:
(a) contacting GPCR135 or an active fragment thereof with a test compound and with a labeled relaxin3 or an active fragment thereof;
(b) determining the amount of the labeled relaxin3 or active fragment thereof that binds to the GPCR135 or active fragment thereof; and
(c) comparing the amount determined in step (b) with a control measurement wherein the GPCR135 or active fragment thereof has been contacted with the labeled relaxin3 or active fragment thereof in the absence of test compound.

27. The method of claim 26, wherein the labeled relaxin3 or active fragment thereof is labeled with ¹²⁵I.

28. A method for identifying a compound that binds GPCR135 and mimics relaxin3, comprising:
(a) contacting a test compound with an assay reagent comprising GPCR135 or an active fragment thereof;
(b) determining a biological activity of the GPCR135 or active fragment thereof; and
(c) comparing the result determined in step (b) with that of a control measurement wherein the GPCR135 or an active fragment thereof was contacted with relaxin3 or an active fragment thereof in the absence of the test compound.

29. The method of claim 28, wherein the GPCR135 or active fragment thereof is expressed from the surface of a recombinant cell.

30. The method of claim 28, wherein the GPCR135 or active fragment thereof is within an isolated cell membrane preparation.


SEQUENCE LISTING (28 sequences)

SEQ ID NO: 1 - DNA, 40 bp, Artificial
  Primer
acagctcgaggccaccatgcagatggccgatgcagccacg

SEQ ID NO: 2 - DNA, 39 bp, Artificial
  Primer
acatcatctagatcagtaggcagagctgctgggcagcag

SEQ ID NO: 3 - DNA, 45 bp, Artificial
  Primer
acgatactcgaggccaccatgcaggtggcttctgcaacccccgcg

SEQ ID NO: 4 - DNA, 41 bp, Artificial
  Primer
actagatctagatcagtaggcagagctactagggagcaggt

SEQ ID NO: 5 - DNA, 47 bp, Artificial
  Primer
acgatactcgaggccaccatgcccaaagcgcacctgagcatgcaagt

SEQ ID NO: 6 - DNA, 41 bp, Artificial
  Primer
acgatatctagatcagtaggcagagctgctagggagaaggt

SEQ ID NO: 7 - DNA, 45 bp, Artificial
  Primer
acgatactcgaggccaccatgcaagtggcttctgcaaccaccgca

SEQ ID NO: 8 - DNA, 1410 bp, Homo sapiens
atgcagatggccgatgcagccacgatagccaccatgaataaggcagcaggcggggacaagctagcagaactcttcagtctggtcccggaccttctggaggcggccaacacgagtggtaacgcgtcgctgcagcttccggacttgtggtgggagctggggctggagttgccggacggcgcgccgccaggacatcccccgggcagcggcggggcagagagcgcggacacagaggcccgggtgcggattctcatcagcgtggtgtactgggtggtgtgcgccctggggttggcgggcaacctgctggttctctacctgatgaagagcatgcagggctggcgcaagtcctctatcaacctcttcgtcaccaacctggcgctgacggactttcagtttgtgctcaccctgcccttctgggcggtggagaacgctcttgacttcaaatggcccttcggcaaggccatgtgtaagatcgtgtccatggtgacgtccatgaacatgtacgccagcgtgttcttcctcactgccatgagtgtgacgcgctaccattcggtggcctcggctctgaagagccaccggacccgaggacacggccggggcgactgctgcggccggagcctgggggacagctgctgcttctcggccaaggcgctgtgtgtgtggatctgggctttggccgcgctggcctcgctgcccagtgccattttctccaccacggtcaaggtgatgggcgaggagctgtgcctggtgcgtttcccggacaagttgctgggccgcgacaggcagttctggctgggcctctaccactcgcagaaggtgctgctgggcttcgtgctgccgctgggcatcattatcttgtgctacctgctgctggtgcgcttcatcgccgaccgccgcgcggcggggaccaaaggaggggccgcggtagccggaggacgcccgaccggagccagcgcccggagactgtcgaaggtcaccaaatcagtgaccatcgttgtcctgtccttcttcctgtgttggctgcccaaccaggcgctcaccacctggagcatcctcatcaagttcaacgcggtgcccttcagccaggagtatttcctgtgccaggtatacgcgttccctgtgagcgtgtgcctagcgcactccaacagctgcctcaaccccgtcctctactgcctcgtgcgccgcgagttccgcaaggcgctcaagagcctgctgtggcgcatcgcgtctccttcgatcaccagcatgcgccccttcaccgccactaccaagccggagcacgaggatcaggggctgcaggccccggcgccgccccacgcggccgcggagccggacctgctctactacccacctggcgtcgtggtctacagcggggggcgctacgacctgctgcccagcagctctgcctactga

SEQ ID NO: 9 - DNA, 1419 bp, Mouse
atgcaggtggcttctgcaacccccgcggccaccgtgaggaaagcagctgcgggtgatgagctctcagaattcttcgctctgaccccagacttgctggaagtggccaacgccagcggcaatgcgtcgctgcagcttcaggatctgtggtgggagctggggctagagttgccagacggtgcggcgcctgggcatcctccgggtggcggcggggcagagagcacagacactgaggccagggtacggatcctcatcagcgcggtttactgggtggtttgtgccctgggactggccggcaacctgctggttctctacctgatgaagagcaagcaaggctggcgcaaatcctccatcaacctctttgtcactaacctggcactgactgactttcagttcgtgctcactctgcccttttgggctgtggagaacgcactagacttcaagtggcccttcggcaaggccatgtgtaagatcgtgtccatggtgacatccatgaacatgtacgccagcgtcttcttcctcactgctatgagcgtggcgcgctaccactcggtggcctcggctctcaagagccatcggacccgagggcgtggccgtggcgactgctgcggccagagcttgagggagagctgctgtttttcagccaaggtgctgtgtgggttgatctgggcttcggctgcgctggcctcgctgcccaatgccattttttccaccaccatcagggtgttgggtgaggagctctgcctcatgcactttccagacaagctactgggctgggacaggcagttctggctgggtttgtaccacctgcagaaggtgctgctgggcttcctgctgccgctgagcatcatcagtctgtgttacctgttgcttgtgcgcttcatctccgaccgtcgcgtagttgggacaacagatgcagtaggagcagcagcagcgcctgggggaggcctgagtacagccagcgctaggagacgctccaaggtcaccaagtcggtgaccatcgtcgtcctctccttcttcctgtgttggctgcccaaccaggcgcttaccacctggagcatcctcatcaagttcaacgccgtgcccttcagccaggagtactttcagtgccaagtgtacgcgttcccagtcagcgtgtgcctggcgcactccaacagctgcctcaacccgatcctctactgcttagtgcgccgcgagttccgcaaggcgctcaagaacctgctgtggcggatagcctcgccttcgctcaccaacatgcgccctttcaccgccaccaccaagccagaacctgaagatcacgggctgcaggccctggcgccgcttaatgctgctgccgaacctgacctgatctactatccacccggtgtggtggtctacagcgggggtcgctacgacctgctccctagtagctctgcctactga

SEQ ID NO: 10 - DNA, 1431 bp, Rat
atgcccaaagcgcacctgagcatgcaagtggcttctgcaaccaccgcagcccccatgagtaaggcagctgcgggtgatgagctctccggattcttcggcctgatcccagacttgctggaggttgccaacaggagcagcaatgcgtcgctgcagcttcaggacttgtggtgggagctggggctggagttgcccgacggtgcggcgcctgggcatcccccgggcagcggtggggcagagagcgcggacacagaggccagggtacggatcctcatcagcgccgtttactgggtggtttgtgccctgggactggctggcaacctgctggttctctacctgatgaagagcaaacagggctggcgcaaatcctccattaacctctttgtcactaacctggcgctgactgactttcagtttgtgctcactctgcccttctgggcggtggagaacgcactagatttcaagtggccctttggcaaggccatgtgtaagatcgtatctatggtgacatccatgaacatgtatgccagcgtcttctttctcactgctatgagtgtggcgcgctaccactcggtggcctcagctctcaagagccatcggacccgcgggcatggccgtggcgactgctgcggccagagcttgggggagagctgctgtttctcagccaaggtgctgtgtggattgatctgggcttctgccgcgatagcttcgctgcccaatgtcattttttctaccaccatcaatgtgttgggcgaggagctgtgcctcatgcactttccggacaagctcctgggttgggaccggcagttctggctgggtttgtaccacctgcagaaggtgctgctgggcttcctgctgccgctgagcatcatcagtttgtgttacctgttgctcgtgcgcttcatctccgaccgccgcgtagtggggacaacggatggagcaacagcgcctggggggagcctgagtacagccggcgctcggagacgctccaaggtcaccaagtcggtgaccatcgtagtcctttccttcttcttatgttggctgcccaaccaagcgctcaccacctggagcatcctcatcaagttcaacgtagtgcccttcagtcaggagtactttcagtgccaagtgtacgcgttcccagtcagcgtgtgcctggcacactccaacagctgcctcaaccccatcctctactgcttagtgcgccgcgagttccgcaaggcgctcaagaacctgctgtggcgtatagcatcgccttcgctcaccagcatgcgccccttcaccgccaccaccaagccagaacctgaagatcacgggctgcaggccctggcgccacttaatgctactgcagagcctgacctgatctactatccacccggtgtggtggtctacagcggaggtcgctacgaccttctccctagcagctctgcctactga

SEQ ID NO: 11 - DNA, 1410 bp, Rat
atgcaagtggcttctgcaaccaccgcagcccccatgagtaaggcagctgcgggtgatgagctctccggattcttcggcctgatcccagacttgctggaggttgccaacaggagcagcaatgcgtcgctgcagcttcaggacttgtggtgggagctggggctggagttgcccgacggtgcggcgcctgggcatcccccgggcagcggtggggcagagagcgcggacacagaggccagggtacggatcctcatcagcgccgtttactgggtggtttgtgccctgggactggctggcaacctgctggttctctacctgatgaagagcaaacagggctggcgcaaatcctccattaacctctttgtcactaacctggcgctgactgactttcagtttgtgctcactctgcccttctgggcggtggagaacgcactagatttcaagtggccctttggcaaggccatgtgtaagatcgtatctatggtgacatccatgaacatgtatgccagcgtcttctttctcactgctatgagtgtggcgcgctaccactcggtggcctcagctctcaagagccatcggacccgcgggcatggccgtggcgactgctgcggccagagcttgggggagagctgctgtttctcagccaaggtgctgtgtggattgatctgggcttctgccgcgatagcttcgctgcccaatgtcattttttctaccaccatcaatgtgttgggcgaggagctgtgcctcatgcactttccggacaagctcctgggttgggaccggcagttctggctgggtttgtaccacctgcagaaggtgctgctgggcttcctgctgccgctgagcatcatcagtttgtgttacctgttgctcgtgcgcttcatctccgaccgccgcgtagtggggacaacggatggagcaacagcgcctggggggagcctgagtacagccggcgctcggagacgctccaaggtcaccaagtcggtgaccatcgtagtcctttccttcttcttatgttggctgcccaaccaagcgctcaccacctggagcatcctcatcaagttcaacgtagtgcccttcagtcaggagtactttcagtgccaagtgtacgcgttcccagtcagcgtgtgcctggcacactccaacagctgcctcaaccccatcctctactgcttagtgcgccgcgagttccgcaaggcgctcaagaacctgctgtggcgtatagcatcgccttcgctcaccagcatgcgccccttcaccgccaccaccaagccagaacctgaagatcacgggctgcaggccctggcgccacttaatgctactgcagagcctgacctgatctactatccacccggtgtggtggtctacagcggaggtcgctacgaccttctccctagcagctctgcctactga

SEQ ID NO: 12 - protein, 469 aa, Homo sapiens
MQMADAATIATMNKAAGGDKLAELFSLVPDLLEAANTSGNASLQLPDLWWELGLGLPDGAPPGHPPGSGGAESADTEARVRILISVVYWVVCALGLAGNLLVLYLMKSMQGWRKSSINLFVTNLALTDFQFVLTLPFWAVENALDFKWPFGKAMCKIVSMVTSMNMYASVFFLTAMSVTRYHSVASALKSHRTRGHGRGDCCGRSLGDSCCFSAKALCVWIWALAALASLPSAIFSTTVKVMGEELCLVRFPDKLLGRDRQFWLGLYHSQKVLLGFVLPLGIIILCYLLLVRFIADRRAAGTKGGAAVAGGRPTGASARRLSKVTKSVTIVVLSFFLCWLPNQALTTWSILIKFNAVPFSQEYFLCQVYAFPVSVCLAHSNSCLNPVLYCLVRREFRKALKSLLRRIASPSITSMRPFTATTKPEHEDQGLQAPAPPHAAAEPDLLYYPPGVVVYSGGRYDLLPSSSAY

SEQ ID NO: 13 - protein, 472 aa, Mouse
MQVASATPAATVRKAAAGDELSEFFALTPDLLEVANASGNASLQLQDLWWELGLELPDGAAPGHPPGGGGAESTDTEARVRILISAVYWVVCALGLAGNLLVLYLMKSKQGWRKSSINLFVTNLALTDFQFVLTLPFWAVENALDFKWPFGKAMCKIVSMVTSMNMYASVFFLTAMSVARYHSVASALKSHRTRGRGRGDCCGQSLRESCCFSAKVLCGLIWASAALASLPNAIFSTTIRVLGEELCLMHFPDKLLGWDRQFWLGLYHLQKVLLGFLLPLSIISLCYLLLVRFISDRRVVGTTDAVGAAAAPGGGLSTASARRRSKVTKSVTIVVLSFFLCWLPNQALTTWSILIKFNAVPFSQEYFQCQVYAFPVSVCLAHSNSCLNPILYCLVRREFRKALKNLLWRIASPSLTNMRPFTATTKPEPEDHGLQALAPLNAAAEPDLIYYPPGVVVYSGGRYDLLPSSSAY

SEQ ID NO: 14 - protein, 476 aa, Rat
MPKAHLSMQVASATTAAPMSKAAAGDELSGFFGLIPDLLEVANRSSNASLQLQDLWWELGLELPDGAAPGHPPGSGGAESADTEARVRILISAVYWVVCALGLAGNLLVLYLMKSKQGRRKSSINLFVTNLALTDFQFVLTLPFWAVENALDFKWPFGKAMCKIVSMVTSMNMYASVFFLTAMSVARYHSVASALKSHRTRGHGRGDCCGQSLGESCCFSAKVLCGLIWASAAIASLPNVIFSTTINVLGEELCLMHFPDKLLGWDRQFWLGLYHLQKVLLGFLLPLSIISLCYLLLVRFISDRRVVGTTDGATAPGGSLSTAGARRRSKVTKSVTIVVLSFFLCWLPNQALTTWSILIKFNVVPFSQEYFQCQVYAFPVSVCLAHSNSCLNPILYCLVRREFRKALKNLLWRIASPSLTSMRPFTATTKPEPEDHGLQALAPLNATAEPDLIYYPPGVVVYSGGRYDLLPSSSAY

SEQ ID NO: 15 - protein, 469 aa, Rat
MQVASATTAAPMSKAAAGDELSGFFGLIPDLLEVANRSSNASLQLQDLWWELGLELPDGAAPGHPPGSGGAESADTEARVRILISAVYWVVCALGLAGNLLVLYLMKSKQGWRKSSINLFVTNLALTDFQFVLTLPFWAVENALDFKWPFGKAMCKIVSMVTSMNMYASVFFLTAMSVARYHSVASALKSHRTRGHGRGDCCGQSLGESCCFSAKVLCGLIWASAAIASLPNVIFSTTINVLGEELCLMHFPDKLLGWDRQFWLGLYHLQKVLLGFLLPLSIISLCYLLLVRFISDRRVVGTTDGATAPGGSLSTAGARRRSKVTKSVTIVVLSFFLCWLPNQALTTWSILIKFNVVPFSQEYFQCQVYAFPVSVCLAHSNSCLNPILYCLVRREFRKALKNLLWRIASPSLTSMRPFTATTKPEPEDHGLQALAPLNATAEPDLIYYPPGVVVYSGGRYDLLPSSSAY

SEQ ID NO: 16 - protein, 17 aa, Porcine
DVLAGLSSNKWGSKSEI

SEQ ID NO: 17 - protein, 19 aa, Porcine
RASPYGVKLGREFIRAVIF

SEQ ID NO: 18 - DNA, 45 bp, Artificial
  Primer
acgatcgtcgacgccaccatggccaggtacatgctgctgctgctc

SEQ ID NO: 19 - DNA, 41 bp, Artificial
  Primer
acgataaagcttctagcaaaggctactgatttcacttttgc

SEQ ID NO: 20 - DNA, 52 bp, Artificial
  Primer
acgatagaattcgatgacgacgataagcgggcagcgccttacggggtcaggc

SEQ ID NO: 21 - DNA, 44 bp, Artificial
  Primer
actataggatccctagcaaaggctactgatttcacttttgctac

SEQ ID NO: 22 - DNA, 102 bp, Artificial
  Oligonucleotide
ctgcaggccgccatgctgaccgcagcgttgctgagctgtgccctgctgctggcactgcctgccacgcgaggagactacaaggacgacgatgacaaggaattc

SEQ ID NO: 23 - DNA, 40 bp, Artificial
  Primer
acgatactgcaggccgccatgctgaccgcagcgttgctga

SEQ ID NO: 24 - DNA, 45 bp, Artificial
  Primer
cagccaggacatctcgtcggccccgaagaaccccaggggttcctt

SEQ ID NO: 25 - DNA, 46 bp, Artificial
  Primer
ggttcttcggggccgacgagatgtcctggctggcctttccagcagc

SEQ ID NO: 26 - DNA, 44 bp, Artificial; identical to SEQ ID NO: 21
  Primer

SEQ ID NO: 27 - DNA, 44 bp, Artificial
  Primer
gactagaagcttgccaccatggagctgaggccctggttgctatg

SEQ ID NO: 28 - DNA, 40 bp, Artificial
  Primer
gacgatagcggccgcagtgggctcatcagagggcgctctg